(19) United States
(12) Patent Application Publication
(10) Pub. No.: US 2016/0040132 A1
(43) Pub. Date: Feb. 11, 2016

(54) THREE-DIMENSIONAL BIOPRINTED PANCREATIC TUMOR MODEL

(71) Applicants: Rosalie Sears, Portland, OR (US); Brittany Allen-Petersen, Portland, OR (US); Ellen Langer, Portland, OR (US)

(72) Inventors: Rosalie Sears, Portland, OR (US); Brittany Allen-Petersen, Portland, OR (US); Ellen Langer, Portland, OR (US)

(73) Assignee: OREGON HEALTH & SCIENCE UNIVERSITY, PORTLAND, OR (US)

(21) Appl. No.: 14/820,307

(22) Filed: Aug. 6, 2015

Related U.S. Application Data
(60) Provisional application No. 62/034,044, filed on Aug. 6, 2014.

(51) Int. Cl. C12N 5/071

(52) U.S. Cl. CPC C12N 5/0697; C12N 2513/00; C12N 2503/02

(57) ABSTRACT

Described are three-dimensional bioprinted pancreatic tumor tissue structures that are multilayer, multicellular three-dimensional structures generated with pancreatic cancer cells surrounded by cell types known to be found in the pancreatic tumor microenvironment.

### BACKGROUND

Pancreatic cancer often has a poor prognosis, even when diagnosed early. Pancreatic cancer typically spreads rapidly and is seldom detected in its early stages, which is a major reason why it is a leading cause of cancer death. Signs and symptoms may not appear until pancreatic cancer is quite advanced and complete surgical removal is no longer possible.

The National Cancer Institute at the National Institutes of Health estimates that 46,420 new cases of pancreatic cancer will be diagnosed in 2014. From 2004 to 2010, the percentage of pancreatic cancer patients surviving beyond five years was 6.7%. Clearly new therapeutics that effectively target pancreatic cancers are needed and as a result, new models that simulate pancreatic tumors are needed to effectively evaluate potential therapeutics.

### SUMMARY

In addition to the genetic events that drive pancreatic adenocarcinoma (PDA) progression, there is also a large expansion of the pancreatic tumor microenvironment, which significantly contributes to tumor progression and therapeutic resistance. Current experimental testing systems do not accurately recapitulate the spatial organization and composition of the pancreatic tumor microenvironment, resulting in a discrepancy between in vitro experimental results and clinical results.

Culturing pancreatic tumor cells in two-dimensional systems has widely been used in the development and characterization of new drugs; however, it is clear that two-dimensional systems do not accurately recapitulate the stromal microenvironment that plays such a prominent role in pancreatic cancer. Similarly, culturing cells in three-dimensional systems by plating on a layer of extracellular matrix (ECM) still does not take into account the cellular complexity and/or spatial organization of tumors, possibly altering the degree of heterogeneity and oncogenic signaling. In addition to overcoming these barriers, three-dimensional PDA can be generated using primary patient tissue, allowing for the analysis of patient drug response within a timeline that could significantly impact patient treatment options.

Accordingly, described herein are three-dimensional bioprinted pancreatic tumor tissue structures that are multilayer, multicellular three-dimensional structures generated with pancreatic cancer cells surrounded by cell types known to be found in the pancreatic tumor microenvironment.

Advantages of the three-dimensional, bioprinted pancreatic cancer tissues described herein include providing a rapid and reproducible model to explore the unique biology of PDA tumors using human cells and identify novel therapeutic compounds for PDA patients.

In one aspect, disclosed herein are three-dimensional, engineered, pancreatic tumor models comprising: stromal microenvironment—the stromal microenvironment comprising pancreatic stellate cells and endothelial cells—and tumor tissue, the tumor tissue comprising pancreatic cancer cells, the tumor tissue encased in the stromal microenvironment to form the three-dimensional, engineered, pancreatic tumor model; provided that the stromal microenvironment and the tumor tissue were bioprinted. In some embodiments, the tumor model is substantially free of pre-formed scaffold. In some embodiments, the pancreatic cancer cells comprise a human pancreatic cancer cell line. In some embodiments, the pancreatic cancer cells comprise disassociated primary patient tumor. In other embodiments, the pancreatic cancer cells comprise disassociated patient-derived xenograft tumor. In additional embodiments, the stromal microenvironment can also comprise cancer associated fibroblasts and immune cells.

In another aspect, disclosed herein are methods of fabricating a three-dimensional, engineered, pancreatic tumor model, the method comprising: preparing a stromal bio-ink, the stromal bio-ink comprising pancreatic stellate cells and endothelial cells; preparing a tumor bio-ink, the tumor bio-ink comprising pancreatic cancer cells; bioprinting the stromal bio-ink and the tumor bio-ink such that the tumor bio-ink is encased in the stromal bio-ink and in contact with the stromal bio-ink on all sides; and maturing the deposited bio-ink in a cell culture media to allow the cells to cohere to form a three-dimensional, engineered, biological tumor model. The bio-ink can also comprise immune cells and cancer associated fibroblasts. In some embodiments, the pancreatic cancer cells comprise a human pancreatic cancer cell line. In other embodiments, the pancreatic cancer cells comprise disassociated primary patient tumor. In yet other embodiments, the pancreatic cancer cells comprise disassociated patient-derived xenograft tumor. In some embodiments, the maturing has a duration of about 10 days or about 5 days. In some embodiments, the stromal bio-ink further comprises a hydrogel. In some embodiments, the tumor bio-ink further comprises a hydrogel. In further embodiments, maturing the deposited bio-ink in a cell culture media removes the hydrogel. In still further embodiments, the tumor bio-ink comprises endothelial cells (including 10%-35% endothelial cells).

In yet another aspect, disclosed herein are methods of identifying a therapeutic agent for pancreatic cancer in an individual, the method comprising: preparing a stromal bio-ink, the stromal bio-ink comprising pancreatic stellate cells and endothelial cells; preparing a tumor bio-ink, the tumor bio-ink comprising primary pancreatic cancer cells from the individual; bioprinting the stromal bio-ink and the tumor bio-ink such that the tumor bio-ink is encased in the stromal bio-ink and in contact with the stromal bio-ink on all sides; maturing the deposited bio-ink in a cell culture media to allow the cells to cohere to form a three-dimensional, engineered, pancreatic tumor model; applying a candidate therapeutic agent to the pancreatic tumor model; measuring viability of the pancreatic cancer cells; and selecting a therapeutic agent for the individual based on the measured viability of the pancreatic cancer cells. In some embodiments, the maturing has a duration of about 10 days or about 5 days. In some embodiments, the stromal bio-ink further comprises a hydrogel. In some embodiments, the tumor bio-ink further comprises a hydrogel. In further embodiments, maturing the deposited bio-ink in a cell culture media removes the hydrogel.

In yet another aspect, disclosed herein are arrays of three-dimensional, engineered, pancreatic tumor models, each pancreatic tumor model comprising: stromal microenvironment, the stromal microenvironment comprising pancreatic stellate cells and endothelial cells; and tumor tissue, the tumor tissue comprising pancreatic cancer cells, the tumor tissue encased in the stromal microenvironment to form the three-dimensional, engineered, pancreatic tumor model; provided that the stromal microenvironment and the tumor tissue were bioprinted; provided that the array is adapted for use in a high throughput assay. In some embodiments, the assay is a drug screening assay, the drug screening assay to determine the safety and/or efficacy of a drug therapy. In a particular embodiment, the array comprises a plurality of three-dimensional, engineered, pancreatic tumor models wherein each pancreatic tumor model is deposited (e.g., bioprinted) into a well of a multi-well plate to form the array.

### BRIEF DESCRIPTION OF THE DRAWINGS

Some of the drawings herein are better understood when presented in color, which is not available in patent application publications. However, Applicants consider the color drawings to be part of the original disclosure and reserve the right to present color versions of the drawings herein in later proceedings.

FIG. 1 shows a non-limiting example of a three-dimensional, bioprinted pancreatic tumor model described herein.

FIG. 2 shows a non-limiting example of an array of three-dimensional, bioprinted pancreatic tumor model; in this case, an array of pancreatic tumor models cultured on transwell inserts in multi-well plates.

FIG. 3A shows a microgram depicting a primary patient pancreatic tumor.

FIG. 3B shows a non-limiting, exemplary microgram depicting a three-dimensional, bioprinted pancreatic tumor model described herein.

FIG. 4A shows a microgram depicting a primary patient pancreatic tumor.

FIG. 4B shows a non-limiting, exemplary microgram depicting a three-dimensional, bioprinted pancreatic tumor model described herein; in this case, a pancreatic tumor model including dissociated primary pancreatic adenocarcinoma tumor cells encased in a stromal mixture containing primary human pancreatic stellate cells and human umbilical vein endothelial cells after 10 days of maturation.

FIG. 5A shows a microgram depicting a primary patient pancreatic tumor; in this case, a tumor stained for CK8/18 and vimentin.

FIG. 5B shows a microgram depicting a primary patient pancreatic tumor; in this case, a tumor stained for e-cadherin and KI67.

FIG. 5C shows a non-limiting, exemplary microgram depicting a bioprinted avatar tumor; in this case, a tumor stained for CK8/18 and vimentin.

FIG. 5D shows a non-limiting, exemplary microgram depicting a bioprinted avatar tumor; in this case, a tumor stained for e-cadherin and KI67.

FIG. 6 is an exemplary micrograms depicting a bioprinted pancreatic adenocarcinoma model; in this case, the model comprises PanF45 cells bioprinted into human pancreatic stellate cells with HUVEC cells and demonstrating CK8/18 positive colonies at Day 10 post-printing.

FIG. 7A shows a non-limiting, exemplary microgram depicting a bioprinted pancreatic adenocarcinoma model that was subsequently dissociated and stained for CK8/18 and vimentin; in this case, a model comprising PanF4NSG5 cells bioprinted into human pancreatic stellate cells with endothelial cells and demonstrating CK8/18 positive colonies at Day 7 post-printing.

FIG. 7B shows a non-limiting, exemplary microgram depicting a bioprinted pancreatic adenocarcinoma model that was subsequently dissociated and stained for CK8/18 and pS6; in this case, a model comprising PanF4NSG5 cells bioprinted into human pancreatic stellate cells with endothelial cells and demonstrating mTOR signaling at Day 7 post-printing.

FIGS. 8A and B show non-limiting, exemplary micrograms depicting bioprinted pancreatic adenocarcinoma models that were subsequently dissociated and stained with α-SMA stain; in this case, a model comprising PanF4NSG5 cells bioprinted into human pancreatic stellate cells with endothelial cells and demonstrating stellate cell activation at Day 7 post-printing.

FIG. 9A shows a non-limiting, exemplary microgram depicting a bioprinted pancreatic adenocarcinoma model that was subsequently dissociated; in this case, a model comprising PanF4NSG2 cells.

FIG. 9B shows a non-limiting, exemplary microgram depicting a bioprinted pancreatic adenocarcinoma model that was subsequently dissociated; in this case, a model comprising PanF4NSG5 cells.

FIGS. 10A and B show non-limiting, exemplary micrograms depicting bioprinted pancreatic adenocarcinoma models that were subsequently dissociated and stained for CD31 (and fibronectin in FIG. 10B, left); in this case, a model comprising PanF45 cells bioprinted into human pancreatic stellate cells with HUVEC cells and demonstrating endothelial networks at Day 6 post-printing.

FIGS. 11A, B, and C show non-limiting, exemplary micrographs depicting bioprinted pancreatic adenocarcinoma models left untreated (FIG. 11A), treated with 10 μM gemcitabine (FIG. 11B), or treated with 100 μM gemcitabine (FIG. 11C).

FIGS. 12A, 12B, 12C, and 12D show results of the following: A tumor bio-ink was generated by resuspending CD18 pancreatic cancer cells in an extrusion compound at concentration of 300 million cells/mL. A stromal bio-ink was generated by resuspending a mix of stromal cells in an extrusion compound at a density of 200 million cells/mL. The stromal cell mix included 75% primary pancreatic stellate cells and 25% primary HUVECs. The stromal bio-ink and tumor bio-ink were bioprinted such that the tumor bio-ink was completely embedded in the stromal bio-ink and in contact with the stromal bio-ink on all sides in a 2×2×1 mm cube. A pancreatic bioprinted media (50% stellate cell media, 25% HUVEC media, 25% DMEM+10% FBS) was then immediately added to the culture. Structures were allowed to grow for 7 days (with media changes every 24 hours) at which point 3 structures were implanted subcutaneously into NSG mice. Tumors were calipered every 7-10 days and harvested at 1-1.5 cm in diameter and processed into formalin fixed paraffin embedded (FFPE) tissues. FIG. 12A is a plot of the in vivo growth rate of the bioprinted tumors. FIG. 12B is an image of CD18 bioprinted structures at day 7 that were FFPE processed and sectioned at SuM. Representative bioprinted structure depicting immunofluorescence of cancer cells (CK8/18) and stromal cells (vimentin) shown. FIG. 12C is an image of Hematoxylin/Eosin (H/E) staining of the bioprinted tissues on day 7 and FIG. 12D is an H/E stain of xenografted bioprinted tissue on day 57.

FIGS. 13A, 13B and 13C show a 2 mm³ piece of primary pancreatic tumor tissue, resected from a patient with informed written patient consent, was coated in Matrigel and implanted subcutaneously into the flank of a 6-week-old NOD.Cg-Prkdcscid II2rgtm1Wjl/SzJ (NSG) mouse (passage 1). This process was repeated until a passage 5 tumor was achieved. The passage 5 tumor was disassociated in collagenase and the subsequent cells were used to generate bioprinted structures as in FIG. 1. Briefly, a bio-ink of disassociated tumor cells at a density of 150 million cells/mL was surrounded by a stromal stromal bio-ink containing 88% primary pancreatic stellate cells and 12% primary HUVECs. The density of the stromal bio-ink was 200 million cells/mL. Media described in FIGS. 12A, 12B, 12C and 12D was added immediately following the bioprinting process. Structures were allowed to grow for 4 days at which point increasing doses of the chemotherapeutic agent Gemcitabine was added (10, 50, and 100 uM). Fresh drug was added during daily media changes for 3 days and structures harvested on day 7 FIG. 13A H/E stained sections of bioprinted structures treated with Gemcitabine at day 7. FIG. 13B is an image showing immunofluorescence of representative bioprinted structures depicting cancer cells (CK8/18) and stromal cells (vimentin) treated with Gemcitabine. FIG. 13C Top panel is an image of an H/E stained section of a bioprinted structure treated with 50 uM Gemcitabine at day 7. FIG. 13C bottom panel is an image showing immunofluorescence of the same structure as the top), depicting cancer cells (CK8/18) and stromal cells (vimentin) treated with 50 uM of Gemcitabine.

FIGS. 14A and 14B show the results where a patient derived xenograft tumor was minced and plated in DMEM+10% FBS. The subsequent epithelial cells that grew out of the tumor pieces were then maintained as a low passage pancreatic cancer cell line. This cell line was treated for 30 minutes with cell tracker green and used to generate bio-ink similar to FIG. 1, but with 150 million cells/mL. Printing conditions and stromal bio-ink were performed as in FIG. 1. Structures were allowed to grow for 7 days and then processed for FFPE. FIG. 14A is an immunofluorescence image of cells stained for expression of Ki67, a marker of cell proliferation. FIG. 14B is an immunofluorescence image of cells stained for expression of endothelial organization (CD31).

FIGS. 15A, 15B, and 15C show the following: structures generated as described in the legend of FIGS. 14A and 14B were used to determine the effects of small molecule targeted kinase inhibitors in this system. Structures containing the low passage patient derived tumor cells were allowed to grow for 4 days and then treated with 2uMINK128, an mTOR inhibitor for 3 days, with fresh drug added daily. At day 7 structures were processed for FFPE. FIG. 15A is an image of an H/E stained section of bioprinted tissue treated with vehicle or INK128. FIG. 15B is an immunofluorescence image of representative bioprinted structures depicting cancer cells (green) and activation of the mTOR pathway (pS6) in both vehicle and treated structures. FIG. 15C is an immunofluorescence image of the same structure in FIG. 15B, but at higher magnification.

FIG. 16 is an immunofluorescence image of bioprinted structures were generated as described in the legend of FIGS. 12A, 12B, 12C and 12D. Capan1 pancreatic cancer cells were used in the tumor bio-ink. Structures were allowed to grow for 7 days and then processed for FFPE. Shown is an immunofluorescence image of a representative bioprinted structure depicting stromal cells (vimentin) and complex networks of endothelial cells (CD31).

FIGS. 17A, 17B, 17C, and 17D show the results where CD18 pancreatic cancer cells were infected with a lentivirus (pLV416G oFLT2A mCherry) to drive expression of Firefly Luciferase and mCherry under the control of the hEF1a promoter. Infected cells were selected with G418 and these pools were then flow sorted to obtain a population with high expression of mCherry. These CD18-mCfLcells were bioprinted as above using a stromal bioink consisting of 75% primary pancreatic stellate cells and 25% primary HUVECs at a total cell concentration of 200 million cells/mL. The tumor bioink consisted of 75% CD18-mCfL and 25% HUVECs at a total cell concentration of 150 million cells/mL. In this experiment, all cells were resuspended in an extrusion compound that contains alginate, and after printing, structures were crosslinked for 72 hrs, and then treated for 24 hours with lyase to reverse the crosslinks. On day 4, structures were either left untreated or were treated with Gemcitabine at 10, 50 or 100 uM. Media was changed each day. Structures were analyzed on day 10 on an IVIS Lumina XRMS (Perkin Elmer) to assess mCherry fluorescence. For luciferase, structures were treated with 150 ug/mL D-Luciferin 20 min prior to assessing flux. FIG. 17A is a set of images showing differences in mCherry in the presence of the indicated amounts of Gemcitabine. FIG. 17B is a bar graph summarizing results similar to those depicted in FIG. 17A. FIG. 17C is an image showing differences in luciferase flux in the presence of the indicated amounts of gemcitabine. FIG. 17D is a bar graph summarizing results similar to those depicted in FIG. 17D.

FIG. 18 is an image of CD18-mCfL cells that were printed as described in the legend to FIGS. 17A, 17B, 17C and 17D. Structures were crosslinked immediately after printing. On day 2, structures were left untreated or were treated daily with 10 ng/mL TGFb. 72 hours after printing, structures were treated with lyase to reverse the crosslinks. Shown are images from the IVIS Lumina XRMS (Perkin Elmer) to assess mCherry fluorescence to determine localization of tumor cells within the structures on day 10.

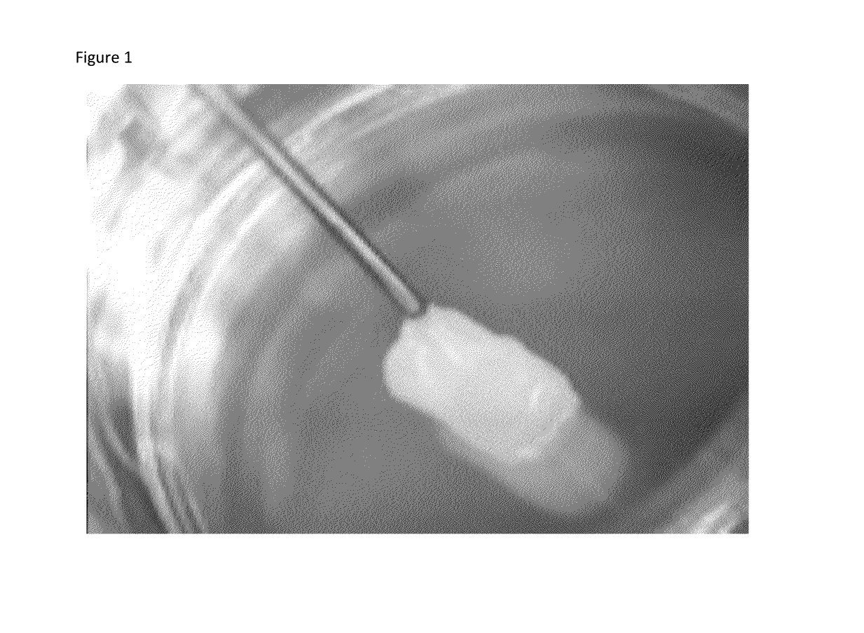

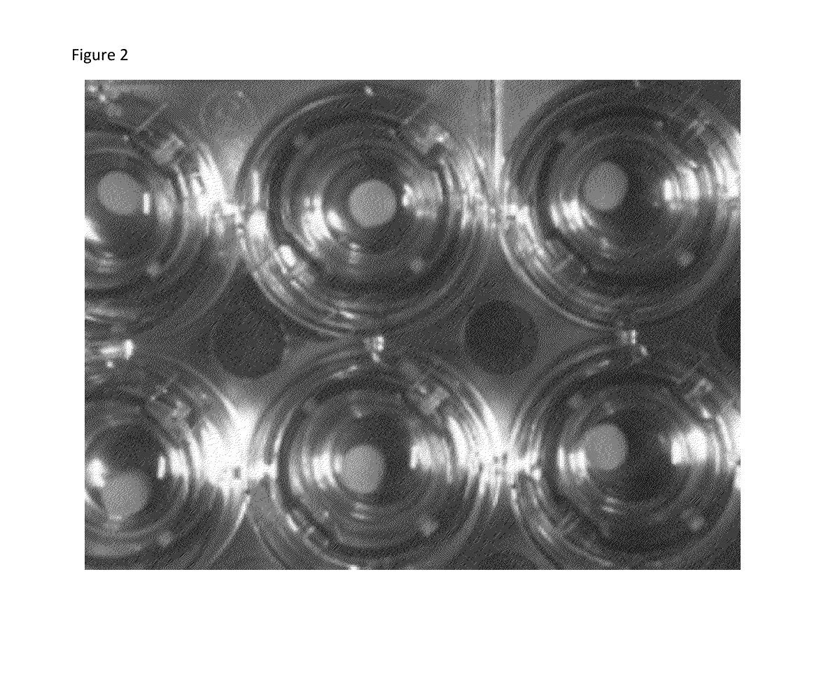

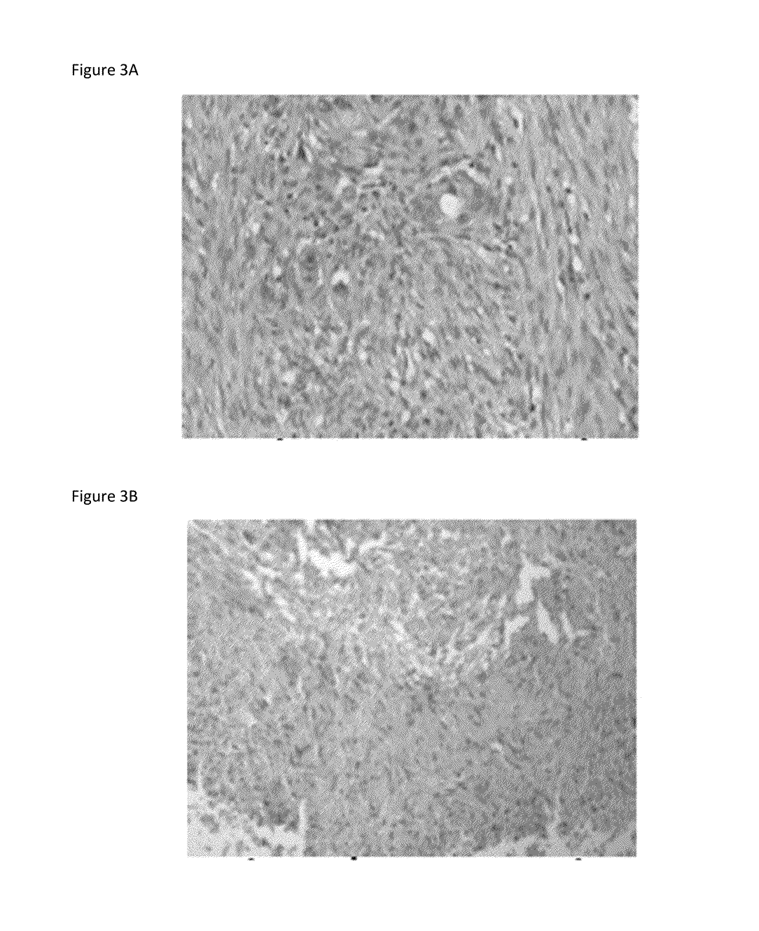

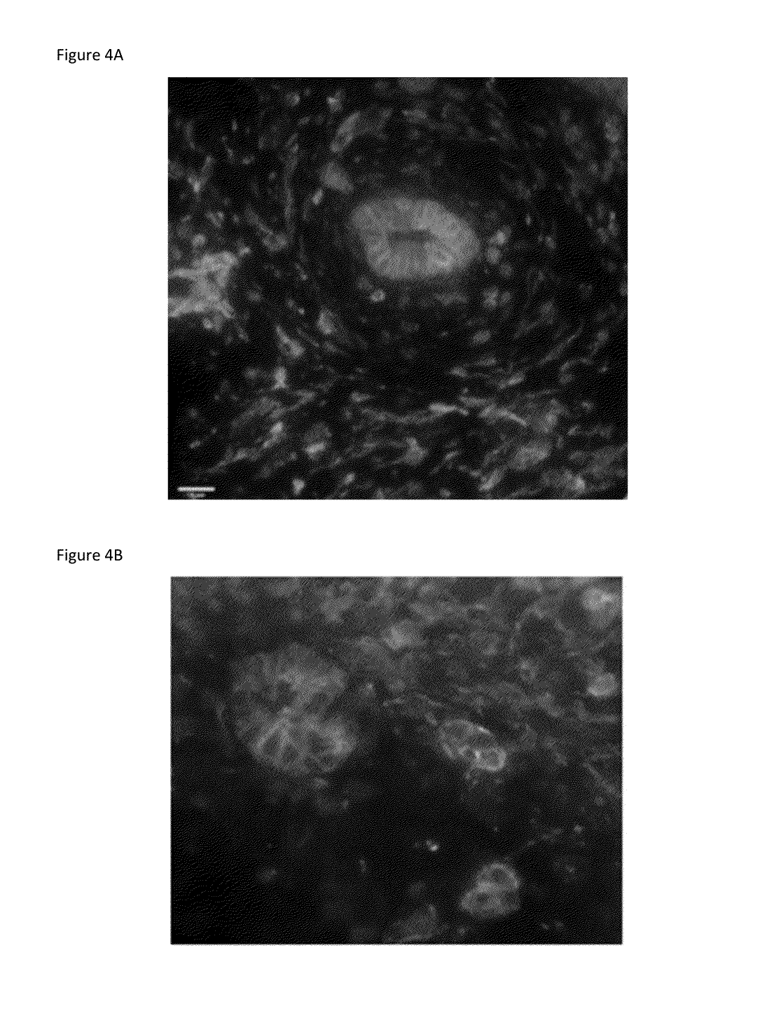

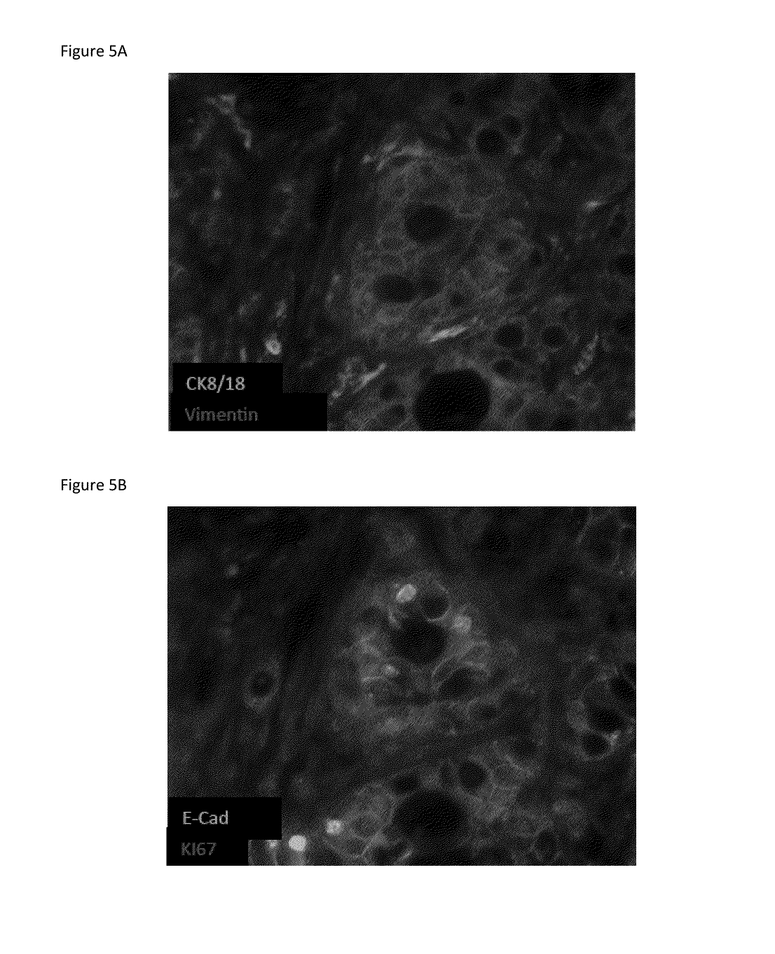

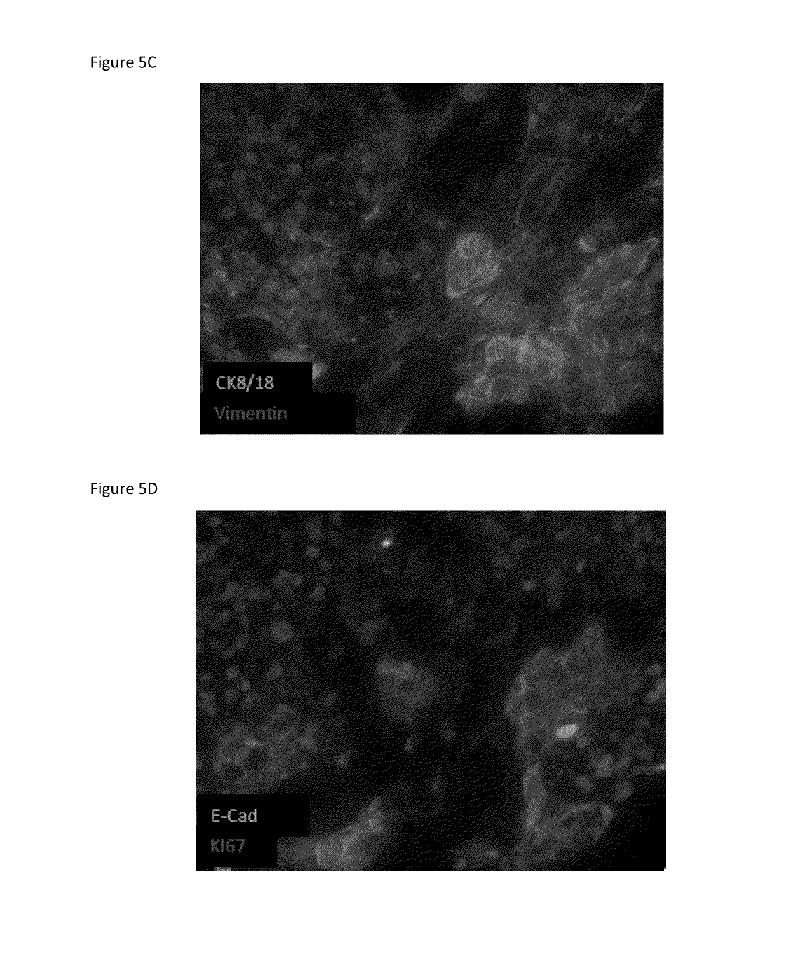

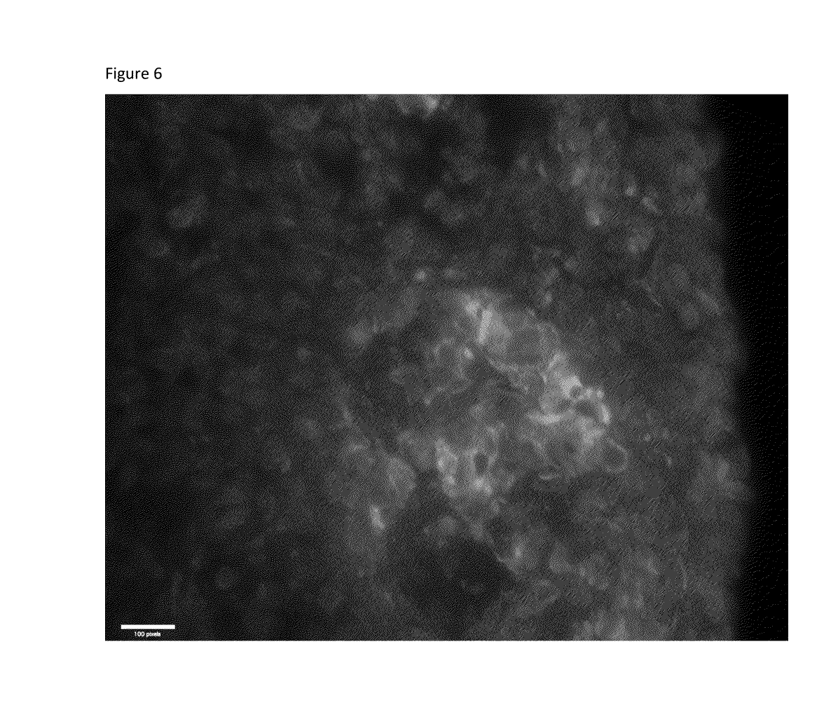

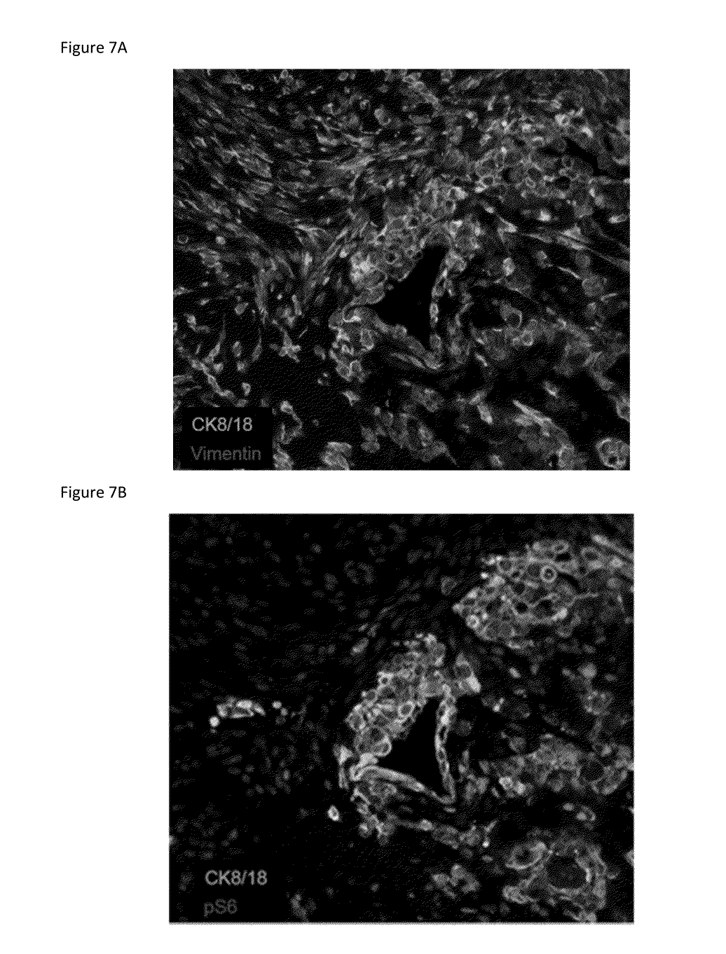

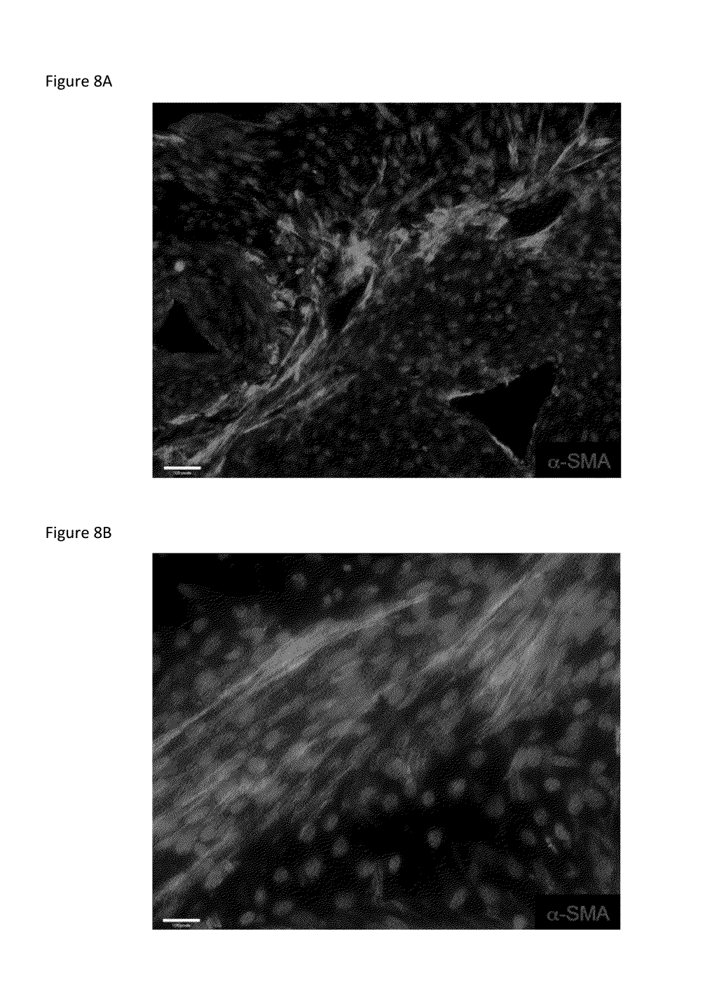

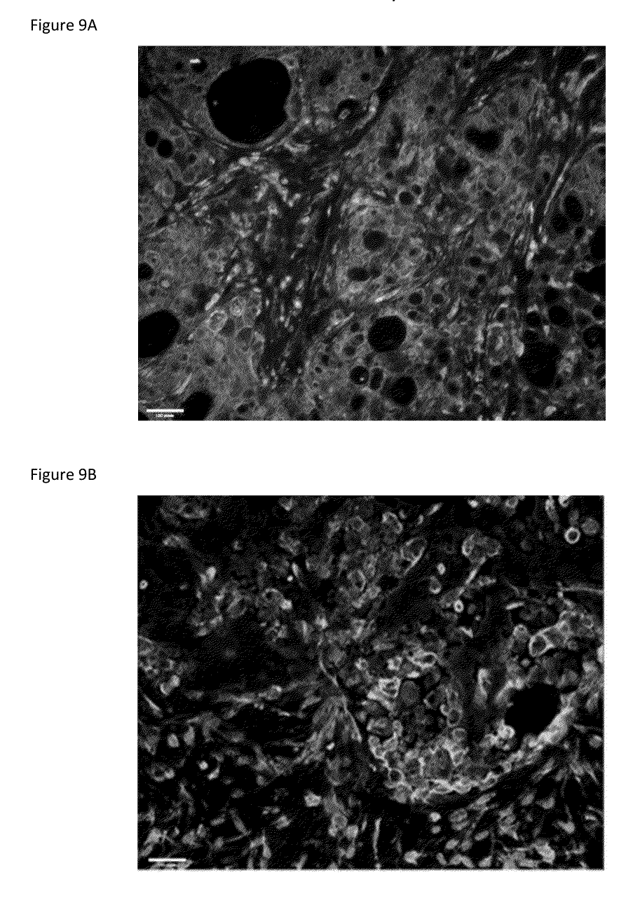

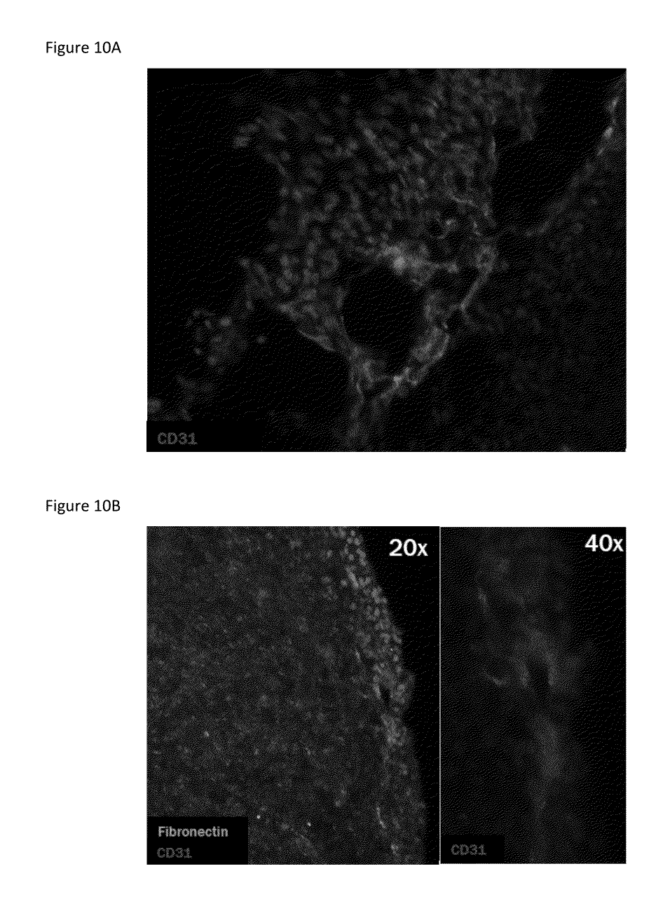

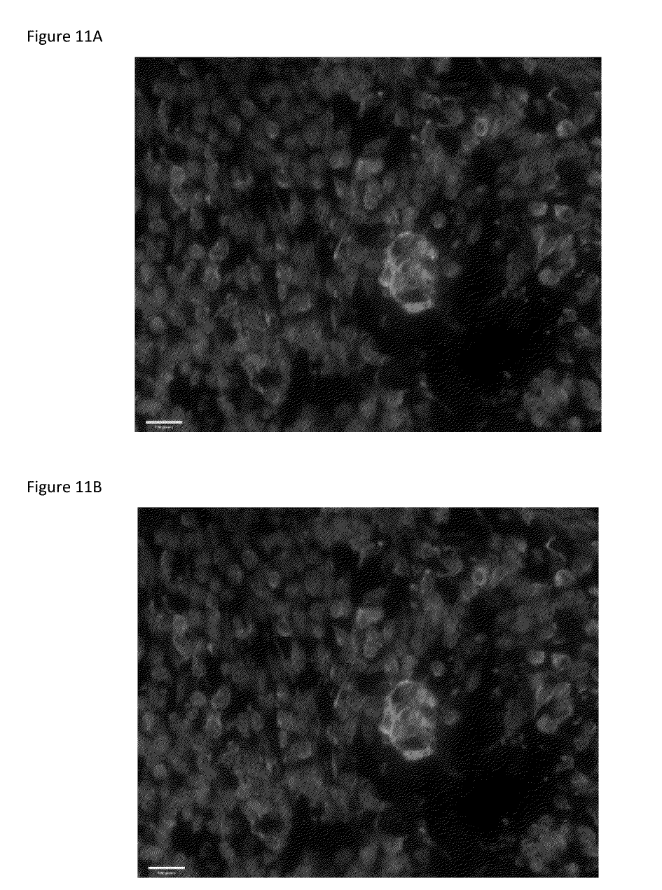

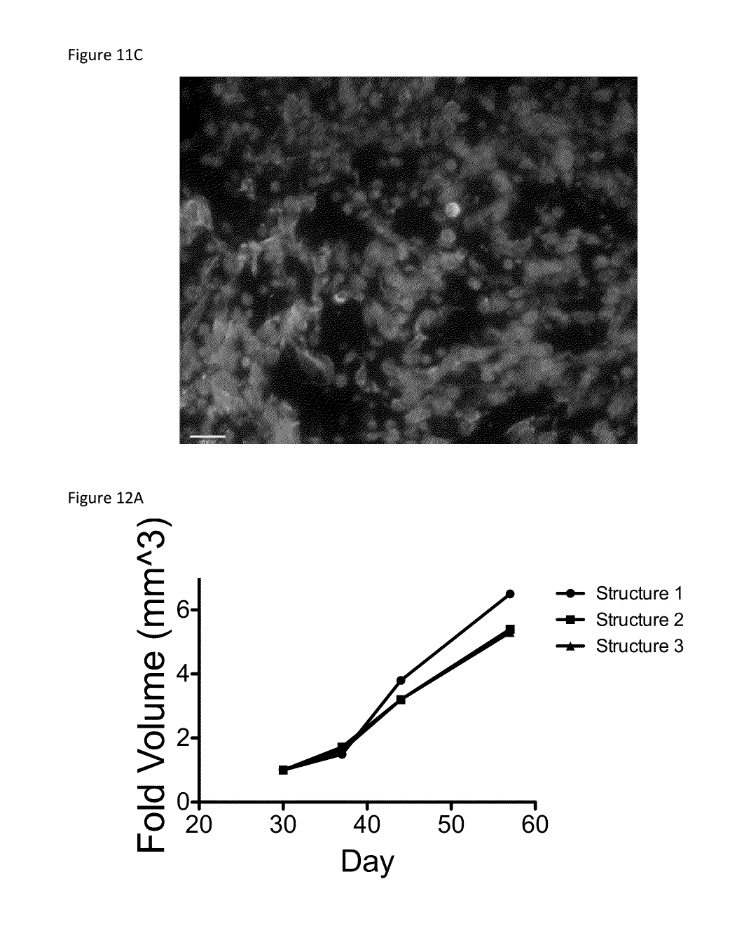

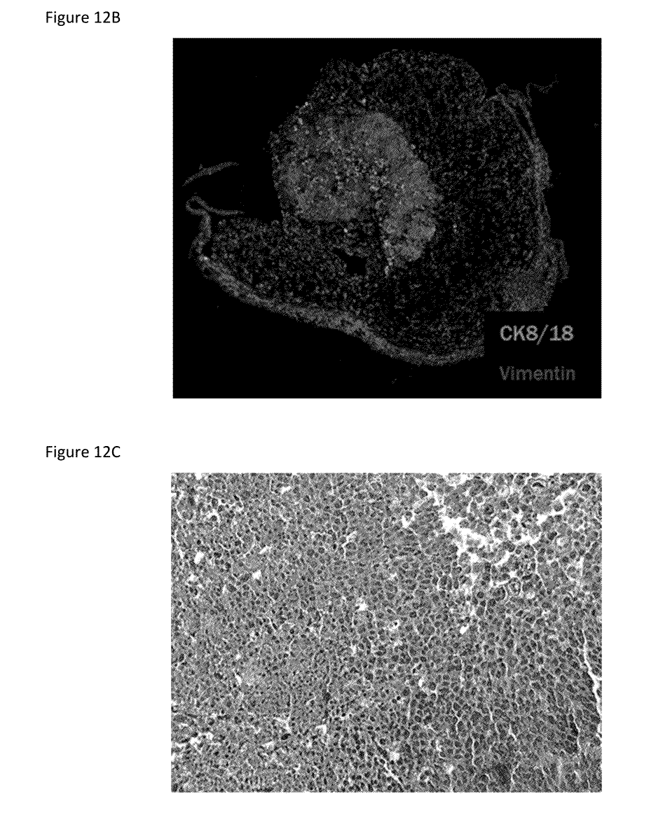

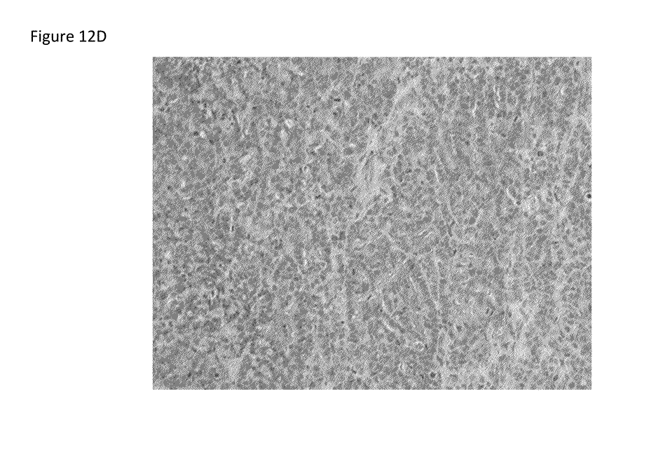

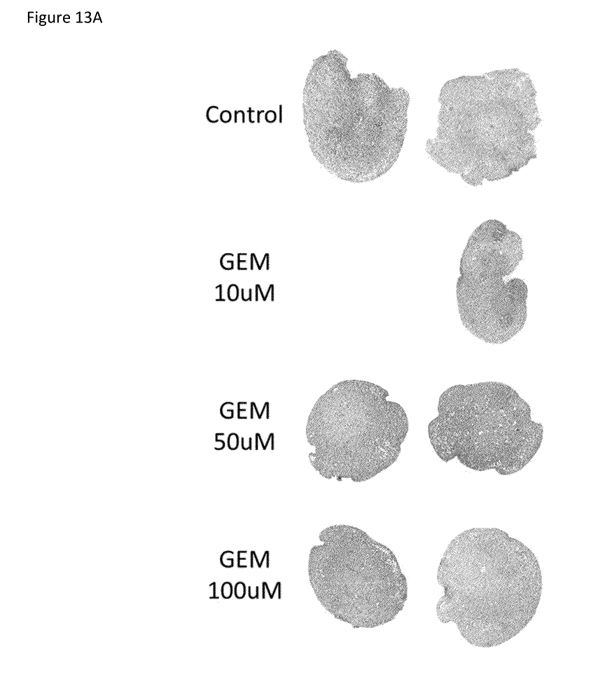

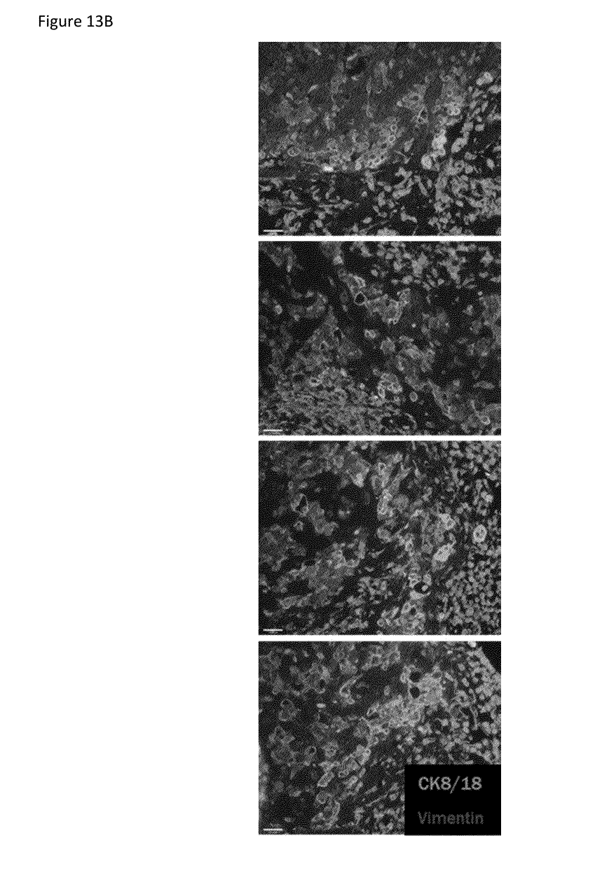

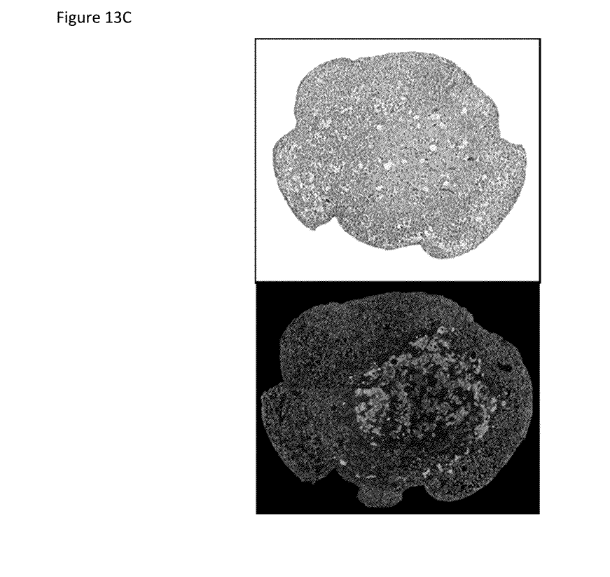

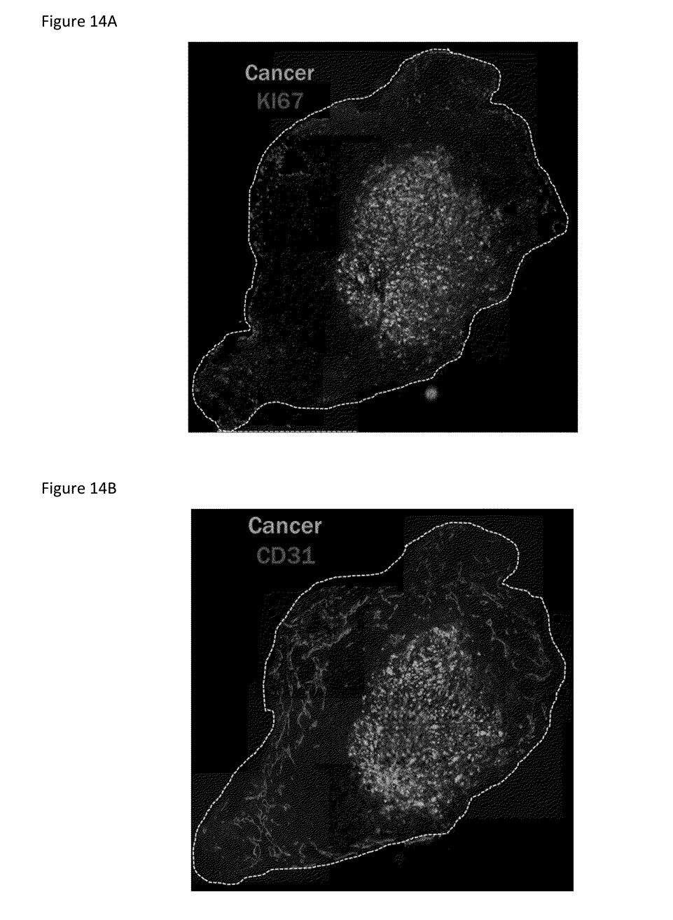

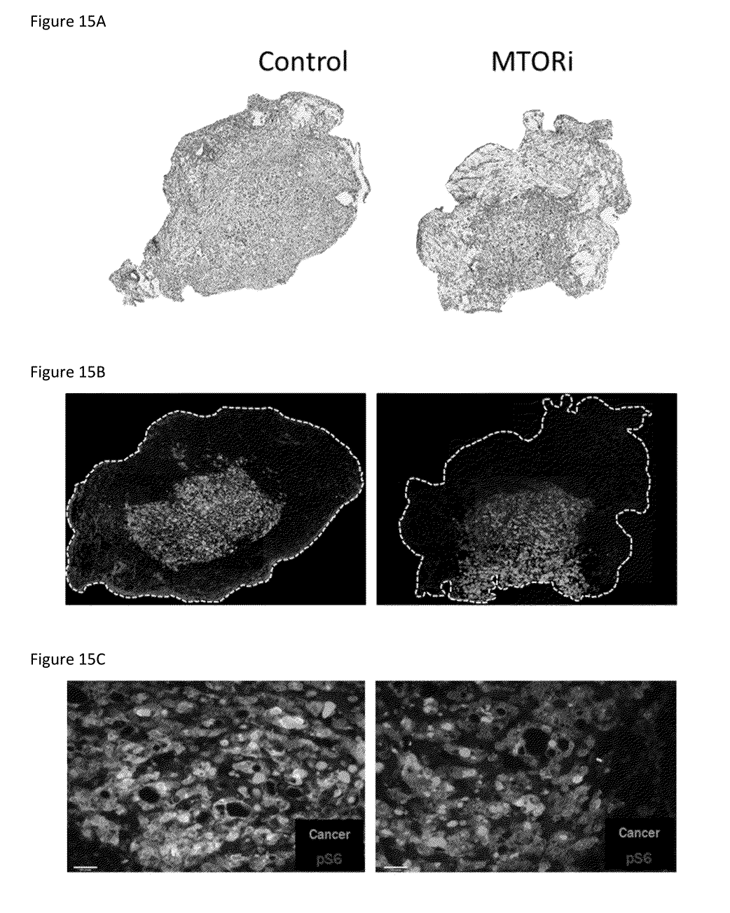

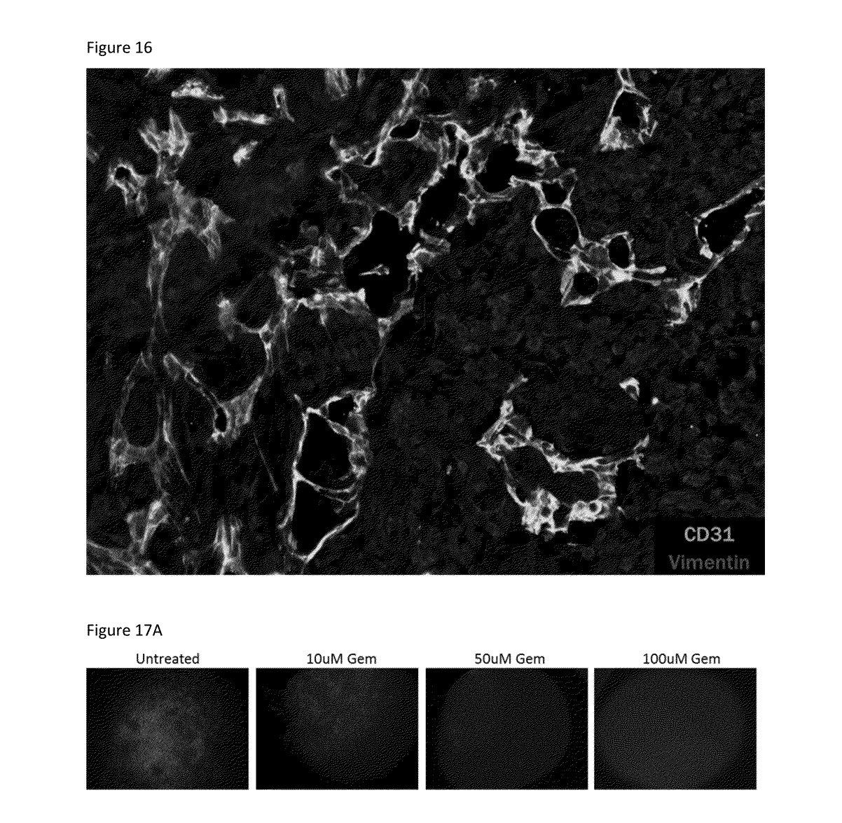

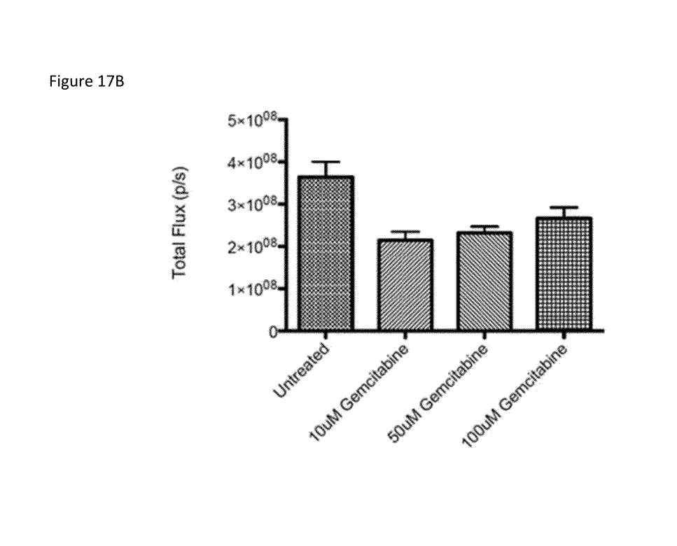

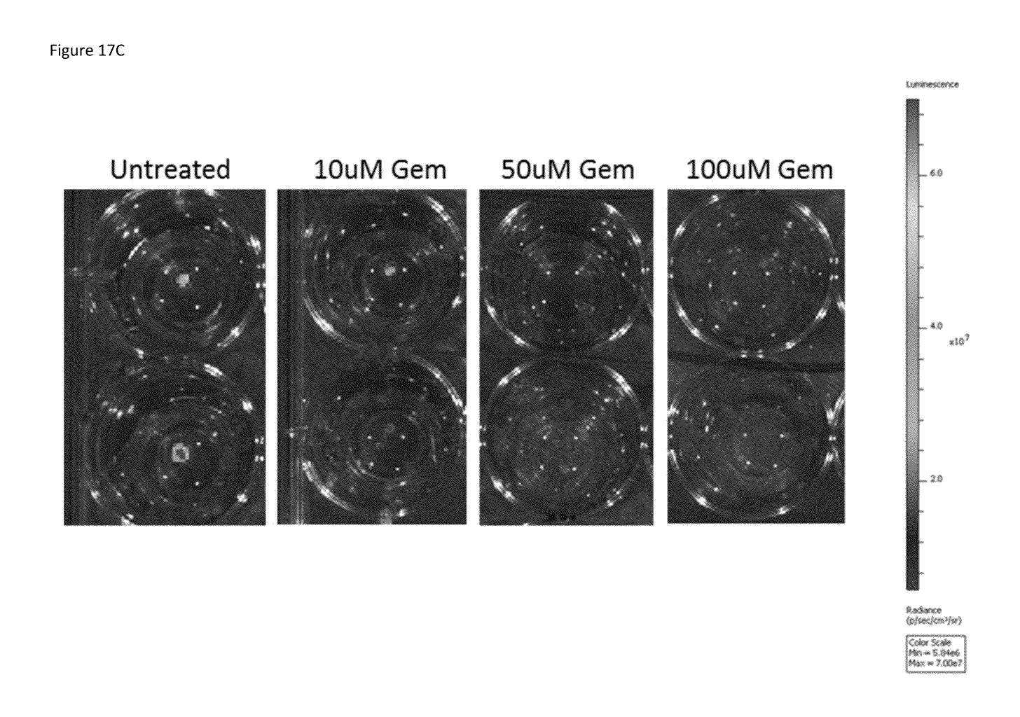

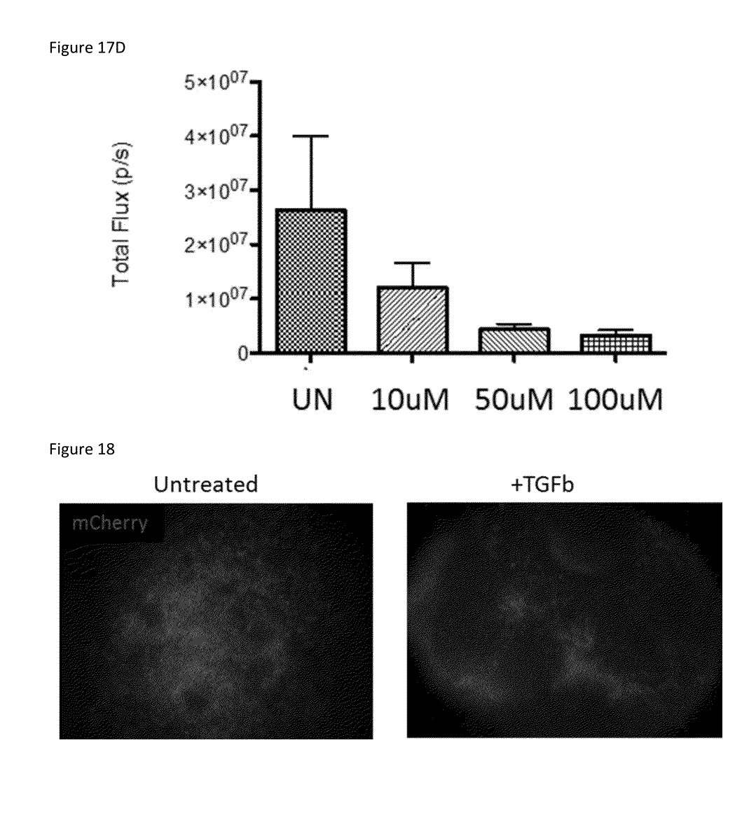

### DETAILED DESCRIPTION

Described herein, in certain embodiments, are three-dimensional, engineered, pancreatic tumor models comprising: stromal microenvironment, the stromal microenvironment comprising pancreatic stellate cells and endothelial cells; and tumor tissue, the tumor tissue comprising pancreatic cancer cells, the tumor tissue encased in the stromal microenvironment to form the three-dimensional, engineered, pancreatic tumor model; provided that the stromal microenvironment and the tumor tissue were bioprinted.

Also described herein, in certain embodiments, are methods of fabricating a three-dimensional, engineered, pancreatic tumor model, the method comprising: preparing a stromal bio-ink, the stromal bio-ink comprising pancreatic stellate cells and endothelial cells; preparing a tumor bio-ink, the tumor bio-ink comprising pancreatic cancer cells; bioprinting the stromal bio-ink and the tumor bio-ink such that the tumor bio-ink is encased in the stromal bio-ink and in contact with the stromal bio-ink on all sides; and maturing the deposited bio-ink in a cell culture media to allow the cells to cohere to form a three-dimensional, engineered, biological tumor model. In some examples, the tumor bio-ink comprises 150 million cells per ml. In some examples the stromal bio-ink comprises 55%-90% pancreatic stellate cells and 10%-35% endothelial cells (such as HUVEC cells) at a density of at least 200 million cells per ml. In still further embodiments, the tumor bio-ink comprises endothelial cells such as HUVEC cells at 10-35% of the total number of cells in the tumor bio-ink.

Also described herein, in certain embodiments, are methods of identifying a therapeutic agent for pancreatic cancer in an individual, the method comprising: preparing a stromal bio-ink, the stromal bio-ink comprising pancreatic stellate cells and endothelial cells; preparing a tumor bio-ink, the tumor bio-ink comprising primary pancreatic cancer cells from the individual; bioprinting the stromal bio-ink and the tumor bio-ink such that the tumor bio-ink is encased in the stromal bio-ink and in contact with the stromal bio-ink on all sides; maturing the deposited bio-ink in a cell culture media to allow the cells to cohere to form a three-dimensional, engineered, pancreatic tumor model; applying a candidate therapeutic agent to the pancreatic tumor model; measuring viability of the pancreatic cancer cells; and selecting a therapeutic agent for the individual based on the measured viability of the pancreatic cancer cells.

Also described herein, in certain embodiments, are arrays of three-dimensional, engineered, pancreatic tumor models, each pancreatic tumor model comprising: stromal microenvironment, the stromal microenvironment comprising pancreatic stellate cells and endothelial cells; and tumor tissue, the tumor tissue comprising pancreatic cancer cells, the tumor tissue encased in the stromal microenvironment to form the three-dimensional, engineered, pancreatic tumor model; provided that the stromal microenvironment and the tumor tissue were bioprinted; provided that the array is adapted for use in a high throughput assay.

### CERTAIN DEFINITIONS

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

As used herein, “array” means a scientific tool including an association of multiple elements spatially arranged to allow a plurality of tests to be performed on a sample, one or more tests to be performed on a plurality of samples, or both.

As used herein, “assay” means a procedure for testing or measuring the presence or activity of a substance (e.g., a chemical, molecule, biochemical, protein, hormone, or drug, etc.) in an organic or biologic sample (e.g., cell aggregate, tissue, organ, organism, etc.).

As used herein, “bio-ink” means a liquid, semi-solid, or solid composition for use in bioprinting. In some embodiments, bio-ink comprises cell solutions, cell aggregates, cell-comprising gels, multicellular bodies, or tissues. In some embodiments, the bio-ink additionally comprises non-cellular materials that provide specific biomechanical properties that enable bioprinting. In some embodiments the bio-ink comprises an extrusion compound.

As used herein, “bioprinting” means utilizing three-dimensional, precise deposition of cells (e.g., cell solutions, cell-containing gels, cell suspensions, cell concentrations, multicellular aggregates, multicellular bodies, etc.) via methodology that is compatible with an automated or semi-automated, computer-aided, three-dimensional prototyping device (e.g., a bioprinter). The bio-inks and tumor models described herein were bioprinted with a Novogen Bioprinter® from Organovo, Inc. (San Diego, Calif.).

As used herein, “scaffold” refers to synthetic scaffolds such as polymer scaffolds and porous hydrogels, non-synthetic scaffolds such as pre-formed extracellular matrix layers, dead cell layers, and decellularized tissues, and any other type of pre-formed scaffold that is integral to the physical structure of the engineered tissue and not able to be removed from the tissue without damage/destruction of said tissue. In further embodiments, decellularized tissue scaffolds include decellularized native tissues or decellularized cellular material generated by cultured cells in any manner; for example, cell layers that are allowed to die or are decellularized, leaving behind the ECM they produced while living. The term “scaffoldless,” therefore, is intended to imply that scaffold is not an integral part of the engineered tissue at the time of use, either having been removed or remaining as an inert component of the engineered tissue. “Scaffoldless” is used interchangeably with “scaffold-free” and “free of pre-formed scaffold.”

As used herein, “stroma” refers to the connective, supportive framework of a biological cell, tissue, or organ.

As used herein, “tissue” means an aggregate of cells.

### Bioprinting

In some embodiments, at least one component of the engineered pancreatic tumor tissues, constructs, or an array thereof, is bioprinted. In further embodiments, bioprinted constructs are made with a method that utilizes a rapid prototyping technology based on three-dimensional, automated, computer-aided deposition of cells, including cell solutions, cell suspensions, cell-comprising gels or pastes, cell concentrations, multicellular bodies (e.g., cylinders, spheroids, ribbons, etc.), and, optionally, confinement material onto a biocompatible support surface (e.g., composed of hydrogel and/or a porous membrane) by a three-dimensional delivery device (e.g., a bioprinter). As used herein, in some embodiments, the term “engineered,” when used to refer to tissues or constructs means that cells, cell solutions, cell suspensions, cell-comprising gels or pastes, cell concentrates, multicellular aggregates, and layers thereof are positioned to form three-dimensional structures by a computer-aided device (e.g., a bioprinter) according to a computer script. In further embodiments, the computer script is, for example, one or more computer programs, computer applications, or computer modules including executable instructions. In still further embodiments, three-dimensional tissue structures form through the post-printing fusion of cells or multicellular bodies which, in some cases, is similar to self-assembly phenomena in early morphogenesis.

While a number of methods are available to arrange cells, cell aggregates, and cell-containing materials on a biocompatible surface to produce a three-dimensional structure, including manual placement, positioning by an automated, computer-aided machine such as a bioprinter is advantageous. Advantages of delivery of cells, cell aggregates, and cell-containing materials with this technology include rapid, accurate, and reproducible placement of cells or multicellular bodies to produce constructs exhibiting planned or pre-determined orientations or patterns of cells, cell aggregates and/or layers thereof with various compositions. Advantages also include assured high cell density, while minimizing cell damage.

In some embodiments, the method of bioprinting is continuous and/or substantially continuous. A non-limiting example of a continuous bioprinting method is to dispense a bio-ink (e.g. tumor and/or stromal cells, tumor and/or stromal cells combined with an excipient or extrusion compound, or aggregates of cells) from a bioprinter via a dispense tip (e.g., a syringe, needle, capillary tube, etc.) connected to a reservoir of bio-ink. In further non-limiting embodiments, a continuous bioprinting method is to dispense bio-ink in a repeating pattern of functional units as in, for example, an array. In various embodiments, a repeating functional unit has any suitable geometry, including, for example, circles, squares, rectangles, triangles, polygons, and irregular geometries, thereby resulting in one or more tissue layers with planar geometry achieved via spatial patterning of distinct bio-inks and/or void spaces. In further embodiments, a repeating pattern of bioprinted function units comprises a layer and a plurality of layers are bioprinted adjacently (e.g., stacked) to form an engineered tissue with laminar geometry. In various embodiments, 2, 3, 4, 5, 6, 7, 8, 9, 10, 11, 12, 13, 14, 15, or more layers are bioprinted adjacently (e.g., stacked) to form an engineered tissue. In further embodiments, one or more layers of a tissue with laminar geometry also has planar geometry.

In some embodiments, continuous bioprinting facilitates printing larger tissues from a large reservoir of bio-ink, optionally using a syringe mechanism. Continuous bioprinting is also a convenient way to co-print spatially-defined boundaries, using an extrusion compound, a hydrogel, a polymer, bio-ink, or any printable material that is capable of retaining its shape post-printing; wherein the boundaries that are created are optionally filled in via the bioprinting of one or more bio-inks, thereby creating a mosaic tissue with spatially-defined planar geometry.

In some embodiments, methods in continuous bioprinting involve optimizing and/or balancing parameters such as print height, pump speed, robot speed, or combinations thereof independently or relative to each other. In certain cases, the bioprinter head speed for deposition was 3 mm/s, with a dispense height of 0.5 mm for the first layer and dispense height was increased 0.4 mm for each subsequent layer. In some embodiments, the dispense height is approximately equal to the diameter of the bioprinter dispense tip. Without limitation a suitable and/or optimal dispense distance does not result in material flattening or adhering to the dispensing needle. In various embodiments, the bioprinter dispense tip has an inner diameter of about, 20, 50, 100, 150, 200, 250, 300, 350, 400, 450, 500, 550, 600, 650, 700, 750, 800, 850, 900, 950, 1000 μm, or more, including increments therein. In various embodiments, the bio-ink reservoir of the bioprinter has a volume of about 0.05, 0.1, 0.5, 1, 2, 3, 4, 5, 6, 7, 8, 9, 10, 15, 20, 25, 30, 35, 40, 45, 50, 55, 60, 65, 70, 75, 80, 85, 90, 95, 100 cubic centimeters, or more, including increments therein. The pump speed is, in some cases, suitable and/or optimal when the residual pressure build-up in the system is low. Favorable pump speeds, in some cases, depend on the ratio between the cross-sectional areas of the reservoir and dispense needle with larger ratios requiring lower pump speeds. In some embodiments, a suitable and/or optimal print speed enables the deposition of a uniform line without affecting the mechanical integrity of the material.

The inventions disclosed herein include business methods. In some embodiments, the speed and scalability of the techniques and methods disclosed herein are utilized to design, build, and operate industrial and/or commercial facilities for production of engineered pancreatic tumor tissues and/or pancreatic tumor disease models for use in generation of tools for research and development, such as in vitro assays. In further embodiments, the engineered tissues and/or models and arrays thereof are produced, stored, distributed, marketed, advertised, and sold as, for example, cellular arrays (e.g., microarrays or chips), tissue arrays (e.g., microarrays or chips), and kits for biological assays and high-throughput drug screening. In other embodiments, the engineered tissues and/or models and arrays thereof are produced and utilized to conduct biological assays and/or drug screening as a service.

### Bio-Ink

Disclosed herein, in certain embodiments, are three-dimensional, living tissues, including pancreatic stromal tissues, pancreatic tumor tissues, pancreatic tumor models, arrays thereof, and methods that comprise bioprinting cells. In some embodiments, cells are bioprinted by depositing or extruding bio-ink from a bioprinter. In some embodiments, “bio-ink” includes liquid, semi-solid, or solid compositions comprising a plurality of cells. In some embodiments, bio-ink comprises liquid or semi-solid cell solutions, cell suspensions, or cell concentrations. In further embodiments, a cell solution, suspension, or concentration comprises a liquid or semi-solid (e.g., viscous) carrier and a plurality of cells. In still further embodiments, the carrier is a suitable cell nutrient media, such as those described herein. In some embodiments, bio-ink comprises a plurality of cells that optionally cohere into multicellular aggregates prior to bioprinting. In further embodiments, bio-ink comprises a plurality of cells and is bioprinted to produce a specific planar and/or laminar geometry; wherein cohesion of the individual cells within the bio-ink takes place before, during and/or after bioprinting.

In some embodiments, the bio-ink is produced by collecting a plurality of cells in a fixed volume; wherein the cellular component(s) represent at least about 30% and at most about 100% of the total volume. In some embodiments, bio-ink comprises semi-solid or solid multicellular aggregates or multicellular bodies. In further embodiments, the bio-ink is produced by 1) mixing a plurality of cells or cell aggregates and a biocompatible liquid or gel in a pre-determined ratio to result in bio-ink, and 2) compacting the bio-ink to produce the bio-ink with a desired cell density and viscosity. In some embodiments, the compacting of the bio-ink is achieved by centrifugation, tangential flow filtration (“TFF”), or a combination thereof.

In some embodiments, the bio-inks disclosed herein are characterized by high cellularity by volume, e.g., a high concentration of living cells. In further embodiments, the bio-ink comprise at least about 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400 or more million cells per milliliter of solution. In a particular embodiment, the bio-inks comprise about 150 million to about 300 million cells/mL. In some embodiments, bio-inks that have high cellularity by volume are used to bioprint engineered tissues and constructs with high cell density. In further embodiments, the engineered tissues and constructs are at least about 30, 35, 40, 45, 50, 55, 60, 65, 70, 75, 80 or more percent cells. In still further embodiments the cells making up a pancreatic tumor stromal cell bio-ink are at least 40, 45, 50, 55, 60, 65, 70, 75, 80, 85, 90, or 95 percent or more pancreatic stellate cells and at least 5, 10, 15, 20, 25, 30, 35, 40, 45, 50, 55, or 60 percent or more endothelial cells such as human umbilical vein endothelial cells (HUVEC), including a tumor bio-ink made up of 55%-90% pancreatic stellate cells and 10%-35% endothelial cells, including a tumor bio-ink of 75% pancreatic stellate cells and 25% endothelial cells.

In some embodiments, the compacting of the bio-ink results in a composition that is extrudable, allowing formation of multicellular aggregates or multicellular bodies. In some embodiments, “extrudable” means able to be shaped by forcing (e.g., under pressure) through a nozzle or orifice (e.g., one or more holes or tubes). In some embodiments, the compacting of the bio-ink results from growing the cells to a suitable density. The cell density necessary for the bio-ink will vary with the cells being used and the tissue or organ being produced.

In some embodiments, the cells of the bio-ink are cohered and/or adhered. In some embodiments, “cohere,” “cohered,” and “cohesion” refer to cell-cell adhesion properties that bind cells, multicellular aggregates, multicellular bodies, and/or layers thereof. In further embodiments, the terms are used interchangeably with “fuse,” “fused,” and “fusion.” In some embodiments, the bio-ink additionally comprises support material, cell culture medium (or supplements thereof), extracellular matrix (or components thereof), cell adhesion agents, cell death inhibitors, anti-apoptotic agents, anti-oxidants, extrusion compounds, and combinations thereof.

In some embodiments, the bio-ink comprises pancreatic cancer cells (interchangeably referred to as tumor cells). In further embodiments, the pancreatic cancer cells are cells of one or more cell lines. In other embodiments, the cancer cells are primary cancer cells derived from the tumor of a patient. In further embodiments, the cancer cells are primary cancer cells isolated from a xenograft (e.g. a tumor grown in an immune compromised mouse) prior to bioprinting.

In some embodiments, the bio-ink comprises an extrusion compound (i.e., a compound that modifies the extrusion properties of the bio-ink). Examples of extrusion compounds include, but are not limited to gels, hydrogels, peptide hydrogels, amino acid-based gels, surfactant polyols (e.g., Pluronic F-127 or PF-127), thermo-responsive polymers, hyaluronates, alginates, extracellular matrix components (and derivatives thereof), collagens, gelatin, other biocompatible natural or synthetic polymers, nanofibers, and self-assembling nanofibers. In some embodiments, extrusion compounds are removed by physical, chemical, or enzymatic means subsequent to bioprinting, subsequent to cohesion of the bioprinted cells, or subsequent to maturation of the bioprinted construct.

Suitable hydrogels include those derived from collagen, hyaluronate, hyaluronan, fibrin, alginate, agarose, chitosan, and combinations thereof. In other embodiments, suitable hydrogels are synthetic polymers. In further embodiments, suitable hydrogels include those derived from poly(acrylic acid) and derivatives thereof, poly(ethylene oxide) and copolymers thereof, poly(vinyl alcohol), polyphosphazene, and combinations thereof. In various specific embodiments, the confinement material is selected from: hydrogel, NovoGel®, agarose, alginate, gelatin, Matrigel™, hyaluronan, poloxamer, peptide hydrogel, poly(isopropyl n-polyacrylamide), polyethylene glycol diacrylate (PEG-DA), hydroxyethyl methacrylate, polydimethylsiloxane, polyacrylamide, poly(lactic acid), silicon, silk, or combinations thereof. In some embodiments, hydrogel-based extrusion compounds are crosslinkable gels. In further embodiments, crosslinkable gels include those crosslinkable by chemical means. For example, in some embodiments, suitable hydrogels include alginate-containing crosslinkable hydrogels. In various embodiments, suitable hydrogels comprise about 0.1, 0.5, 1, 2, 3, 4, 5, 6, 7, 8, 9, 10, or more percent alginate. In some embodiments, following bioprinting, constructs are optionally incubated with an agent to chemically crosslink the hydrogel, such as a solution of CaCl₂, in order preserve a bioprinted architecture prior to cohesion of the cells. Further, in some embodiments, the bioprinted constructs are optionally incubated with alginate lyase to enzymatically degrade the hydrogel. In further embodiments, the bioprinted constructs are optionally incubated with alginate lyase at a concentration of about 0.2-0.5 mg/ml to enzymatically degrade the hydrogel.

In some embodiments, suitable hydrogels include gelatin. In various embodiments, suitable hydrogels comprise about 1, 2, 3, 4, 5, 6, 7, 8, 9, 10, 11, 12, 13, 14, 15, 16, 17, 18, 19, 20 or more percent gelatin. In some embodiments, the concentration of gelatin is about 5-15% and the concentration of alginate is about 0.5-5% in the extrusion compound or hydrogel. In a particular embodiment, the concentration of gelatin is 10% and the concentration of alginate is 1% in the extrusion compound or hydrogel.

In some embodiments, hydrogel-based extrusion compounds are thermoreversible gels (also known as thermo-responsive gels or thermogels). In some embodiments, a suitable thermoreversible hydrogel is not a liquid at room temperature. In specific embodiments, the gelation temperature (Tgel) of a suitable hydrogel is about 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., including increments therein. In certain embodiments, the Tgel of a suitable hydrogel is about 10° C. to about 40° C. In further embodiments, the Tgel of a suitable hydrogel is about 20° C. to about 30° C. In some embodiments, the bio-ink (e.g., comprising hydrogel, one or more cell types, and other additives, etc.) described herein is not a liquid at room temperature. In some embodiments, a suitable thermoreversible hydrogel is not a liquid at mammalian body temperature. In specific embodiments, the gelation temperature (Tgel) of a suitable hydrogel is about 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 41° C., 43° C., 44° C., 45° C., 46° C., 47° C., 48° C., 49° C., 50° C., 51° C., 52° C., including increments therein. In certain embodiments, the Tgel of a suitable hydrogel is about 22° C. to about 52° C. In further embodiments, the Tgel of a suitable hydrogel is about 32° C. to about 42° C. In some embodiments, the bio-ink (e.g., comprising hydrogel, one or more cell types, and other additives, etc.) described herein is not a liquid at mammalian body temperature. In specific embodiments, the gelation temperature (Tgel) of a bio-ink described herein is about 10° C., about 15° C., about 20° C., about 25° C., about 30° C., about 35° C., about 40° C., about 45° C., about 50° C., about 55° C., including increments therein.

Polymers composed of polyoxypropylene and polyoxyethylene form thermoreversible gels when incorporated into aqueous solutions. These polymers have the ability to change from the liquid state to the gel state at temperatures maintainable in a bioprinter apparatus. The liquid state-to-gel state phase transition is dependent on the polymer concentration and the ingredients in the solution.

In some embodiments, the viscosity of the hydrogels and bio-inks presented herein is measured by any means described. For example, in some embodiments, an LVDV-II+CP Cone Plate Viscometer and a Cone Spindle CPE-40 are used to calculate the viscosity of the hydrogels and bio-inks. In other embodiments, a Brookfield (spindle and cup) viscometer is used to calculate the viscosity of the hydrogels and bio-inks. In some embodiments, the viscosity ranges referred to herein are measured at room temperature. In other embodiments, the viscosity ranges referred to herein are measured at body temperature (e.g., at the average body temperature of a healthy human).

In further embodiments, the hydrogels and/or bio-inks are characterized by having a viscosity of between about 500 and 1,000,000 centipoise, between about 750 and 1,000,000 centipoise; between about 1000 and 1,000,000 centipoise; between about 1000 and 400,000 centipoise; between about 2000 and 100,000 centipoise; between about 3000 and 50,000 centipoise; between about 4000 and 25,000 centipoise; between about 5000 and 20,000 centipoise; or between about 6000 and 15,000 centipoise.

In some embodiments, the non-cellular components of the bio-ink (e.g., extrusion compounds, etc.) are removed prior to use. In further embodiments, the non-cellular components are, for example, hydrogels, peptide hydrogels, amino acid-based gels, surfactant polyols, thermo-responsive polymers, hyaluronates, alginates, collagens, or other biocompatible natural or synthetic polymers. In still further embodiments, the non-cellular components are removed by physical, chemical, or enzymatic means. In some embodiments, a proportion of the non-cellular components remain associated with the cellular components at the time of use.

### Pre-Formed Scaffold

In some embodiments, disclosed herein are engineered tissues and tumor models that are free or substantially free of any pre-formed scaffold. In further embodiments, “scaffold” refers to synthetic scaffolds such as polymer scaffolds and porous hydrogels, non-synthetic scaffolds such as pre-formed extracellular matrix layers, dead cell layers, and decellularized tissues, and any other type of pre-formed scaffold that is integral to the physical structure of the engineered tissue and/or organ and not removed from the tissue and/or organ. In still further embodiments, decellularized tissue scaffolds include decellularized native tissues or decellularized cellular material generated by cultured cells in any manner; for example, cell layers that are allowed to die or are decellularized, leaving behind the ECM they produced while living.

In some embodiments, the engineered tissues and tumor models (including arrays of the same) do not utilize any pre-formed scaffold, e.g., for the formation of the tissue, any layer of the tissue, or formation of the tissue's shape. As a non-limiting example, the engineered pancreatic tissues of the present disclosure do not utilize any pre-formed, synthetic scaffolds such as polymer scaffolds, pre-formed extracellular matrix layers, or any other type of pre-formed scaffold at the time of manufacture or at the time of use. In some embodiments, the engineered pancreatic tissues are substantially free of any pre-formed scaffolds. In further embodiments, the cellular components of the tissues contain a detectable, but trace or trivial amount of scaffold, e.g., less than 2.0%, less than 1.0%, less than 0.5%, or less than 0.1% of the total composition. In still further embodiments, trace or trivial amounts of scaffold are insufficient to affect long-term behavior of the tissue, or array thereof, or interfere with its primary biological function. In additional embodiments, scaffold components are removed post-printing, by physical, chemical, or enzymatic methods, yielding an engineered tissue that is free or substantially-free of scaffold components.

### Arrays

In some embodiments, disclosed herein are arrays of engineered pancreatic tissues and arrays of engineered tumor models (including pancreatic cancer tumor models). In some embodiments, an “array” is a scientific tool including an association of multiple elements spatially arranged to allow a plurality of tests to be performed on a sample, one or more tests to be performed on a plurality of samples, or both. In some embodiments, the arrays are adapted for, or compatible with, screening methods and devices, including those associated with medium- or high-throughput screening. In further embodiments, an array allows a plurality of tests to be performed simultaneously. In further embodiments, an array allows a plurality of samples to be tested simultaneously. In some embodiments, the arrays are cellular microarrays. In further embodiments, a cellular microarray is a laboratory tool that allows for the multiplex interrogation of living cells on the surface of a solid support. In other embodiments, the arrays are tissue microarrays. In further embodiments, tissue microarrays include a plurality of separate tissues or tissue samples assembled in an array to allow the performance of multiple biochemical, metabolic, molecular, or histological analyses. In some embodiments, the engineered tissues and/or tumor models each exist in a well of a biocompatible multi-well container. In some embodiments, each tissue is placed into a well. In other embodiments, each tissue is bioprinted into a well. In further embodiments, the wells are coated. In various further embodiments, the wells are coated with one or more of: a biocompatible hydrogel, one or more proteins, one or more chemicals, one or more peptides, one or more antibodies, and one or more growth factors, including combinations thereof. In some embodiments, the wells are coated with NovoGel®. In other embodiments, the wells are coated with agarose. In some embodiments, each tissue exists on a porous, biocompatible membrane within a well of a biocompatible multi-well container. In some embodiments, each well of a multi-well container contains two or more tissues.

In some embodiments, the engineered tissues and/or tumor models are secured to a biocompatible surface on one or more sides. Many methods are suitable to secure a tissue to a biocompatible surface. In various embodiments, a tissue is suitably secured to a biocompatible surface, for example, along one or more entire sides, only at the edges of one or more sides, or only at the center of one or more sides. In various further embodiments, a tissue is suitably secured to a biocompatible surface with a holder or carrier integrated into the surface or associated with the surface. In various further embodiments, a tissue is suitably secured to a biocompatible surface with one or more pinch-clamps or plastic nubs integrated into the surface or associated with the surface. In some embodiments, a tissue is suitably secured to a biocompatible surface by cell-attachment to a porous membrane. In some embodiments, the engineered tissues and/or tumor models are held in an array configuration by affixation to a biocompatible surface on one or more sides. In further embodiments, the tissue is affixed to a biocompatible surface on 1, 2, 3, 4, or more sides. In some embodiments, the biocompatible surface any surface that does not pose a significant risk of injury or toxicity to the tissue or an organism contacting the tissue. In further embodiments, the biocompatible surface is any surface suitable for traditional tissue culture methods. Suitable biocompatible surfaces include, by way of non-limiting examples, treated plastics, membranes, porous membranes, coated membranes, coated plastics, metals, coated metals, glass, treated glass, and coated glass, wherein suitable coatings include hydrogels, ECM components, chemicals, proteins, etc., and coatings or treatments provide a means to stimulate or prevent cell and tissue adhesion to the biocompatible surface.

In some embodiments, the arrays of engineered tissues and/or tumor models comprise an association of two or more elements. In various embodiments, the arrays comprise an association of 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 96, 100, 125, 150, 175, 200, 225, 250, 275, 300, 325, 350, 375, 384, 400, 425, 450, 475, 500, or 1536 or more elements, including increments therein. In further embodiments, each element comprises one or more cells, multicellular aggregates, tissues, tumor models, or combinations thereof.

In some embodiments, the arrays of engineered tissues and/or tumor models comprise multiple elements spatially arranged in a pre-determined pattern. In further embodiments, the pattern is any suitable spatial arrangement of elements. In various embodiments, patterns of arrangement include, by way of non-limiting examples, a two-dimensional grid, a three-dimensional grid, one or more lines, arcs, or circles, a series of rows or columns, and the like. In further embodiments, the pattern is chosen for compatibility with medium- or high-throughput biological assay or screening methods or devices.

In various embodiments, the cell types and/or source of the cells used to fabricate one or more tissues or tumor models in an array are selected based on a specific research goal or objective. In further various embodiments, the specific tissues or tumor models in an array are selected based on a specific research goal or objective. In some embodiments, one or more specific engineered tissues are included in an array to facilitate investigation of a particular disease or condition. In some embodiments, one or more specific engineered tissues are included in an array to facilitate investigation of a disease or a condition of a particular subject. In further embodiments, one or more specific engineered tissues within the array are generated with one or more cell types derived from two or more distinct human donors. In some embodiments, each tissue within the array is substantially similar with regard to cell types, sources of cells, layers of cells, ratios of cells, methods of construction, size, shape, and the like. In other embodiments, one or more of the tissues within the array is unique with regard to cell types, sources of cells, layers of cells, ratios of cells, methods of construction, size, shape, and the like.

In some embodiments, each tissue and/or tumor model within the array is maintained independently in culture. In further embodiments, the culture conditions of each tissue within the array are such that they are isolated from the other tissues and cannot exchange media or factors soluble in the media. In other embodiments, two or more individual tissues within the array exchange soluble factors. In further embodiments, the culture conditions of two or more individual tissues within the array are such that they exchange media and factors soluble in the media with other tissues. In various embodiments, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, or more of the tissues within the array, including increments therein, exchange media and/or soluble factors. In other various embodiments, 1, 2, 3, 4, 5, 6, 7, 8, 9, 10, 15, 20, 25, 30, 35, 40, 45, 50, 55, 60, 65, 70, 75, 80, 85, 90, 91, 92, 93, 94, 95, 96, 97, 98, 99, or 100% of the tissues within the array, including increments therein, exchange media and/or soluble factors.

In some embodiments, the three-dimensional, engineered, pancreatic tumor models are engineered by encasing cancer cells in stromal cells. In further embodiments, the stromal cells include primary pancreatic stellate cells and/or primary human umbilical vascular endothelial cells in an inert hydrogel. In some embodiments, the cancer cells printed in the center of the structure include human pancreatic cancer cell lines, disassociated primary patient tumor, and/or disassociated patient derived xenograft tumor.

In some embodiments, the three-dimensional, engineered, pancreatic tumor models are cultured for up to 10 days and demonstrate distinct cell populations detectable by immunohistochemistry (IHC), immunofluorescence (IF), or flow cytometry activated cell sorting (FACS). In some embodiments, the three-dimensional, engineered, pancreatic tumor models demonstrate self organization of CD31 positive endothelial cells, activation of stellate cells as shown by α-SMA positive staining, cancer cell phenotypic differences with alterations in migration and polarization, and differential responses to chemotherapeutic treatments. In further embodiments, the three-dimensional, engineered, pancreatic tumor models demonstrate cell death, proliferation, cell-cell junctions, and an observable border between cancer and stroma. In still further embodiments, the three-dimensional, engineered, pancreatic tumor models demonstrate activation of cell signaling molecules (i.e., phospho-S6) that are known to contribute to pancreatic cancer cell survival.

In some embodiments, the engineered pancreatic tumor models described herein are distinguished from tissues fabricated by prior technologies by virtue of the fact that they are three-dimensional, free of pre-formed scaffolds, consist essentially of cells, and/or have a high cell density (e.g., greater than 30% cellular, greater than 40% cellular, or greater than 50% cellular).

In some embodiments, the three-dimensional, engineered pancreatic tumor models described herein are distinguished from native (e.g., non-engineered) tissues by virtue of the fact that they are non-innervated (e.g., substantially free of nervous tissue), substantially free of a mature circulatory system, including fully-formed vascular networks and related blood cells. For example, in various embodiments, the three-dimensional, engineered pancreatic tumor models are free of plasma, red blood cells, white blood cells, platelets, and the like.

### Multicellular

The three-dimensional, engineered, pancreatic tumor models utilize several human cell types known to contribute to PDA progression (stellate and endothelial cells), all of which can be manipulated prior to bioprinting. This cellular complexity can significantly alter the survival, therapeutic resistance, and cell fate of pancreatic cancer cells, creating a tissue that more closely resembles in vivo tumors.

In vivo many tumors are in continuous contact with stromal microenvironment. In some embodiments, the tissue comprises a stromal microenvironment and a tumor tissue. In some embodiments, the stromal microenvironment completely surrounds the tumor tissue on all sides. In some embodiments, the stromal microenvironment completely surrounds the tumor tissue on all sides, except for where the tumor tissue may be attached to a biocompatible surface. In some embodiments, the stromal microenvironment is in continuous contact with the tumor tissue. In some embodiments, the stromal microenvironment is in at least 90% continuous contact with the tumor tissue. In some embodiments, the stromal microenvironment is in at least 80% continuous contact with the tumor tissue. In some embodiments, the stromal microenvironment is in at least 70% continuous contact with the tumor tissue. In some embodiments, the stromal microenvironment is in at least 60% continuous contact with the tumor tissue.

The tissues and methods disclosed herein exhibit improvements compared to methods that require low cell density for bioprinting, such as ink-jet printing; or methods that require significant tissue growth and formation post-bioprinting, such as techniques that require embedding aggregates in matrix or hydrogel support. In some embodiments, either the stromal microenvironment or the tumor tissue is present at a high density. In some embodiments, either the stromal microenvironment or the tumor tissue is present at a high density immediately after bioprinting. In some embodiments, either the stromal microenvironment or the tumor tissue is present at a high density within 1 hour after bioprinting. In some embodiments, either the stromal microenvironment or the tumor tissue is present at a high density within 6 hours after bioprinting. In some embodiments, either the stromal microenvironment or the tumor tissue is present at a high density within 24 hours after bioprinting. In some embodiments, either the stromal microenvironment or the tumor tissue is at a concentration of at least about 10 million cells per cubic centimeter. In some embodiments, either the stromal microenvironment or the tumor tissue is at a concentration of at least about 20 million cells per cubic centimeter. In some embodiments, either the stromal microenvironment or the tumor tissue is at a concentration of at least about 30 million cells per cubic centimeter. In some embodiments, either the stromal microenvironment or the tumor tissue is at a concentration of at least about 40 million cells per cubic centimeter. In some embodiments, either the stromal microenvironment or the tumor tissue is at a concentration of at least about 50 million cells per cubic centimeter. In some embodiments, either the stromal microenvironment or the tumor tissue is at a concentration of at least about 100 million cells per cubic centimeter. In some embodiments, either the stromal microenvironment or the tumor tissue is at a concentration of at least about 200 million cells per cubic centimeter.

### Three-Dimensional Organization

Culturing cells on plastic or ECM does not allow for the spatial organization of cell types. Cells in three-dimensional PDA structures are able to self organize and form cellular structures, such as endothelial networks, that resemble those found in pancreatic tumors. Additionally, we are able to analyze the border between cancer and stroma, which will give us the unique ability to identify factors that contribute to migration and metastasis.

Many shapes and sizes are suitable for the engineered pancreatic tumor tissues. By way of example, in one embodiment, the engineered pancreatic tumor tissues are bioprinted in form of a sheet. By way of further example, in other embodiments, the engineered pancreatic tissues are bioprinted in form of a cube or block. By way of further example, in another embodiment, the engineered pancreatic tumor tissues are bioprinted in form of a sphere. Finally, in other embodiments, the engineered pancreatic tumor tissues are bioprinted in form of cylinder or ribbon. In some embodiments, the engineered pancreatic tumor tissues are about 250 μm to about 5 mm in their smallest dimension. In some embodiments, the engineered pancreatic tumor tissues are about 250 μm to about 5 mm in their largest dimension. In a particular embodiment, the engineered pancreatic tissues are bioprinted in the form of cubes that are 2 or 3 mm on each side. In such embodiments, the tissues form spheres after a period of maturation in cell culture conditions.

Also described herein, in some embodiments are arrays of three-dimensional, engineered, biological pancreatic tumor tissues that are adapted for use in medium- or high-throughput assays such as drug screening assays, drug discovery assays, drug safety and toxicity assays, drug efficacy assays, and the like. In some embodiments, the arrays are created by depositing an engineered pancreatic tumor tissue into each well of a multi-well plate to form a grid of tissues.

Subsequently, in some embodiments, the methods include preparing a bio-ink, the bio-ink comprising an extrusion composition (such as a hydrogel) and cell types such as pancreatic stellate cells and endothelial cells. In a particular embodiment, the bio-ink comprises, for example 75% pancreatic stellate cells and 25% endothelial cells such as, for example, HUVEC cells. Such a bio-ink can comprise about 200 million cells/ml In further embodiments, the bio-ink comprises about 150 to about 300 million pancreatic tumor cells per mL.

Further, in some embodiments, the methods include depositing the bio-ink onto a biocompatible surface via an automated or semi-automated deposition device such as a bioprinter. In some embodiments, one or more components of the engineered pancreatic tumor tissue is bioprinted. In other embodiments, each component of the engineered pancreatic tumor tissue is bioprinted. In a particular embodiment, the engineered pancreatic tumor tissues are built up, layer by layer, by bioprinting to form a three-dimensional structure. Still further, in some embodiments, the methods include maturing the deposited bio-ink in a cell culture media to allow the cells to cohere to form the three-dimensional, engineered, biological pancreatic tumor tissue. In some embodiments, the cell culture media removes the hydrogel of the bio-ink leaving a substantially cellular construct. In some embodiments, the cell culture media comprises a mixture of media suited to the cell types included in the bio-ink. For example, in a particular embodiment, the cell culture media comprises human endothelial cell media or pancreatic stellate cell media.

The engineered pancreatic tumor tissues described herein have many advantageous uses. For example, cancer cells are optionally introduced into the engineered pancreatic tumor tissues to form pancreatic cancer tumor model. By way of further example, an oncogenic agent is optionally applied to the engineered pancreatic tumor tissues to provide an initiation event in order to generate pancreatic cancer model. By way of still further example, a diseased pancreas is fabricated in order to generate a diseased pancreatic cancer model. Further in this example, the diseased pancreatic tumor tissue is optionally exposed to a pathogen such as one or more viruses (to become viral-loaded) or one or more bacteria (to become bacteria-loaded). Such constructs are useful for research in the field of oncology and for investigation of therapies for the treatment of cancer.

Described herein, in some embodiments are three-dimensional, engineered, biological tumor models comprising stromal tissue and tumor tissue. In some embodiments, the stromal tissue comprises stromal cells. In some embodiments, the tumor tissue comprises cancer cells. The engineered tumor models described herein have a compartmentalized architecture. For example, in some embodiments, the stromal tissue of the tumor model surrounds the tumor tissue. In further embodiments, the stromal tissue of the tumor model surrounds the tumor tissue on, for example, three or more, four or more, five or more, or six or more sides. In still further embodiments, the stromal tissue of the tumor model completely surrounds the tumor tissue such that the tumor tissue is embedded in the stromal tissue to form the engineered tumor model.

Many stromal cells are suitable for inclusion in the engineered tumor models. For example, in various embodiments, the engineered tumor models suitably include one or more of: fibroblasts, endothelial cells, epithelial cells, adipocytes, and immune cells such as macrophages. In various embodiments, the cells are vertebrate cells, mammalian cells, human cells, or combinations thereof. Many cancer cells are suitable for inclusion in the engineered tumor models. For example, in various embodiments, the engineered tumor models suitably include one or more of: cancer cell lines and primary cancer cells excised from a patient tumor. In some embodiments, the tumor tissue further comprises endothelial cells and/or immune cells such as macrophages.

In some embodiments, the cells are bioprinted. In further embodiments, bioprinted stromal cells are cohered to form the engineered stromal tissue. In further embodiments, bioprinted cancer cells are cohered to form the engineered tumor tissue. In still further embodiments, the stromal tissue and the tumor tissue are cohered to form the engineered tumor model. In some embodiments, the engineered tumor models are free or substantially free of pre-formed scaffold at the time of fabrication or the time of use. In some embodiments, the engineered tumor models are non-innervated. In further embodiments, the engineered tumor models lack an intact neural system and/or mature neural tissues. In some embodiments, the engineered tumor models lack an intact vascular system and/or a mature vasculature. In further embodiments, the engineered pancreatic tissues are free of red blood cells.

Also described herein, in some embodiments are arrays of three-dimensional, engineered, biological tumor models that are adapted for use in medium- or high-throughput assays such as drug screening assays, drug discovery assays, drug safety and toxicity assays, drug efficacy assays, and the like. In some embodiments, the arrays are created by depositing an engineered tumor model into each well of a multi-well plate to form a grid of tumor models.

In some embodiments the pancreatic tumor models differ from naturally occurring pancreatic tumors. In some embodiments, the pancreatic tumors lack neuronal innervation. In some embodiments, the pancreatic tumors lack vascularization. In some embodiments, the pancreatic tumors are of a uniform shape. In some embodiments, the uniform shape is spherical, ellipsoid, cuboid, trapezoidal or rhomboidal. In some embodiments, the pancreatic tumors contain transgenic DNA inserted into one or more different cell types. In some embodiments the transgenic DNA encodes a protein from a non-human species. In some embodiments, the transgenic DNA encodes a fluorescent or luminescent protein. In some embodiments the transgenic DNA encodes an antibiotic resistance protein. In certain embodiments one or more of the cell types of the pancreatic tumor model, is a non-human species.

In some embodiments the pancreatic tumor models differ from standard 2D co-culture models. In some embodiments, the pancreatic tumors are greater than 2 cells thick. In some embodiments, the pancreatic tumors are greater than 3 cells thick. In some embodiments, the pancreatic tumors are greater than 5 cells thick. In some embodiments, the pancreatic tumors are greater than 10 cells thick. In some embodiments, the pancreatic tumors are greater than 100 cells thick.

### Assays

In some embodiments, the engineered tissues, including pancreatic tissues, and tumor models, including pancreatic cancer tumor models, described herein are for use in in vitro assays. In some embodiments, an “assay” is a procedure for testing or measuring the presence or activity of a substance (e.g., a chemical, molecule, biochemical, drug, etc.) in an organic or biologic sample (e.g., cell aggregate, tissue, organ, organism, etc.). In further embodiments, assays include qualitative assays and quantitative assays. In still further embodiments, a quantitative assay measures the amount of a substance in a sample.

In various embodiments, the engineered tissues, including pancreatic tissues, and tumor models, including pancreatic cancer tumor models, described herein are for use in, by way of non-limiting examples, image-based assays, measurement of secreted proteins, expression of markers, and production of proteins. In various further embodiments, the engineered tissues, including pancreatic tissues, and tumor models, including pancreatic cancer tumor models, described herein are for use in assays to detect or measure one or more of: molecular binding (including radioligand binding), molecular uptake, activity (e.g., enzymatic activity and receptor activity, etc.), gene expression, protein expression, receptor agonism, receptor antagonism, cell signaling, apoptosis, chemosensitivity, transfection, cell migration, chemotaxis, cell viability, cell proliferation, safety, efficacy, metabolism, toxicity, and abuse liability.

In some embodiments, the engineered tissues, including pancreatic tissues, and tumor models, including pancreatic cancer tumor models, described herein are for use in immunoassays. In further embodiments, immunoassays are competitive immunoassays or noncompetitive immunoassays. In a competitive immunoassay, for example, the antigen in a sample competes with labeled antigen to bind with antibodies and the amount of labeled antigen bound to the antibody site is then measured. In a noncompetitive immunoassay (also referred to as a “sandwich assay”), for example, antigen in a sample is bound to an antibody site; subsequently, labeled antibody is bound to the antigen and the amount of labeled antibody on the site is then measured.

In some embodiments, the engineered tissues, including pancreatic tissues, and tumor models, including pancreatic cancer tumor models, described herein are for use in enzyme-linked immunosorbent assays (ELISA). In further embodiments, an ELISA is a biochemical technique used to detect the presence of an antibody or an antigen in a sample. In ELISA, for example, at least one antibody with specificity for a particular antigen is utilized. By way of further example, a sample with an unknown amount of antigen is immobilized on a solid support (e.g., a polystyrene microtiter plate) either non-specifically (via adsorption to the surface) or specifically (via capture by another antibody specific to the same antigen, in a “sandwich” ELISA). By way of still further example, after the antigen is immobilized, the detection antibody is added, forming a complex with the antigen. The detection antibody is, for example, covalently linked to an enzyme, or is itself detected by a secondary antibody that is linked to an enzyme through bioconjugation.

For example, in some embodiments, an array, microarray, or chip of cells, multicellular aggregates, or tissues is used for drug screening or drug discovery. In further embodiments, an array, microarray, or chip of tissues is used as part of a kit for drug screening or drug discovery. In some embodiments, each vascular wall segment exists within a well of a biocompatible multi-well container, wherein the container is compatible with one or more automated drug screening procedures and/or devices. In further embodiments, automated drug screening procedures and/or devices include any suitable procedure or device that is computer or robot-assisted. In some embodiments, arrays for drug screening assays or drug discovery assays are used to research or develop drugs potentially useful in any therapeutic area. In still further embodiments, suitable therapeutic areas include, by way of non-limiting examples, infectious disease, hematology, oncology, pediatrics, cardiology, central nervous system disease, neurology, gastroenterology, hepatology, urology, infertility, ophthalmology, nephrology, orthopedics, pain control, psychiatry, pulmonology, vaccines, wound healing, physiology, pharmacology, dermatology, gene therapy, toxicology, and immunology.

In some embodiments, arrays for therapy screening assays or therapy discovery assays are used to identify therapies potentially useful in the disease or condition of a particular individual or group of individuals. For example, in some embodiments, the methods described herein include utilizing cells of a particular individual to engineer tissues, disease models, or tumor models. In further embodiments, the methods include applying a candidate therapeutic agent to the tissue or model; measuring viability of the cells; and selecting a therapeutic agent for the individual based on the measured viability of the cells. In still further embodiments, the candidate therapeutic agent is a one or more chemotherapeutic compounds, one or more radiopharmaceutical compounds, radiation therapy, or a combination thereof. Accordingly, disclosed herein are methods of personalizing medicine to an individual or group of individuals.

### Primary Cells

In addition to commercially available primary and immortalized human pancreatic cells, primary tissue from PDA patients can be disassociated and printed into three-dimensional, engineered, pancreatic tumor models that contain the native cell types found in each patient's tumor. The use of primary patient tissue in this system will enable culture of relevant cell types and preservation of tumor heterogeneity, both of which can alter drug response and oncogenic signaling, and thus provide a better model for therapeutic testing.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

What is claimed is:

1. A three-dimensional, engineered, pancreatic tumor model comprising:
a. a stromal microenvironment, the stromal microenvironment comprising pancreatic stellate cells and endothelial cells; and
b. tumor tissue, the tumor tissue comprising pancreatic cancer cells, the tumor tissue encased in the stromal microenvironment to form the three-dimensional, engineered, pancreatic tumor model;
provided that the stromal microenvironment and the tumor tissue were bioprinted.

2. The pancreatic tumor model of claim 1, wherein the model is substantially free of pre-formed scaffold.

3. The pancreatic tumor model of claim 1, wherein the pancreatic cancer cells comprise a human pancreatic cancer cell line, disassociated primary patient tumor, or disassociated patient-derived xenograft tumor.

4. The pancreatic tumor model of claim 1 arranged as a tissue array.

5. The pancreatic tumor model of claim 1 wherein the tumor tissue and the stromal microenvironment are in at least 60% continuous contact.

6. The pancreatic tumor model of claim 1 wherein the stromal microenvironment is at a concentration of at least 10 million cells per cubic centimeter.

7. The pancreatic tumor model of claim 1 wherein the tumor tissue is at a concentration of at least 10 million cells per cubic centimeter.

8. The pancreatic tumor model of claim 1 wherein the pancreatic tumor lacks neuronal innovation or vascularization, wherein the pancreatic tumor has a uniform shape, and wherein the pancreatic tumor comprises transgenic DNA from a human or non-human species.

9. A method of fabricating a three-dimensional, engineered, pancreatic tumor model, the method comprising:
a. preparing a stromal bio-ink, the stromal bio-ink comprising pancreatic stellate cells and endothelial cells
b. preparing a tumor bio-ink, the tumor bio-ink comprising pancreatic cancer cells;
c. bioprinting the stromal bio-ink and the tumor bio-ink such that the tumor bio-ink is encased in the stromal bio-ink and in contact with the stromal bio-ink on all sides; and
d. maturing the deposited bio-ink in a cell culture media to allow the cells to cohere to form a three-dimensional, engineered, biological tumor model.

10. The method of claim 9, wherein the pancreatic cancer cells comprise a human pancreatic cancer cell line, a disassociated primary patient tumor, or a disassociated patient derived xenograft tumor.

11. The method of claim 9 wherein the cell density of the tumor bio-ink is between 150 million and 300 million cells/ml.

12. The method of claim 9, comprising maturing the deposited bio-ink in the cell culture media for at least 10 days.

13. The method of claim 9, wherein the stromal bio-ink and/or the tumor bio-ink further comprises a hydrogel.

14. The method of claim 13, wherein the maturing the deposited bio-ink in a cell culture media removes the hydrogel.

15. The method of claim 9 wherein the stromal bio-ink comprises 55%-90% pancreatic stellate cells and 10%-35% endothelial cells.

16. The method of claim 9 wherein the density of cells in the stromal bio ink is at least 200 million cells/ml.

17. The method of claim 9, wherein the tumor bio-ink further comprises endothelial cells.

18. A method of identifying a therapeutic agent for pancreatic cancer in an individual, the method comprising:
a. fabricating a three-dimensional, engineered, pancreatic tumor model by the method of claim 9;
b. applying a candidate therapeutic agent to the pancreatic tumor model;
c. measuring viability of the pancreatic cancer cells; and
d. selecting a therapeutic agent for the individual based on the measured viability of the pancreatic cancer cells.
